(19) United States
(12) Patent Application Publication
(10) Pub. No.: US 2017/0273610 A1
(43) Pub. Date: Sep. 28, 2017

(54) WEARABLE ELECTROCHEMICAL SENSOR AND METHOD

(71) Applicant: Glucovation, Inc, Carlsbad, CA (US)

(72) Inventors: Jeff T. Suri, Carlsbad, CA (US); Robert Boock, Carlsbad, CA (US); Kenneth San Vicente, San Diego, CA (US)

(21) Appl. No.: 15/731,056

(22) PCT Filed: Mar. 12, 2015
(86) PCT No.: PCT/US2015/020112

§ 371 Date: Apr. 12, 2017

Related U.S. Application Data
(60) Provisional application No. 61/951,667, filed on Mar. 12, 2014.

(51) Int. Cl. A61B 5/1486; A61B 5/00; A61B 5/151; A61B 5/145

(52) U.S. Cl. CPC A61B 5/14865; A61B 5/14532; A61B 5/14546; A61B 5/0004; A61B 5/742; A61B 5/6849; A61B 5/15105; A61B 2560/045; A61B 2560/063

(57) ABSTRACT

An electrochemical sensing system includes a working electrode and a reference electrode, which can at least partially be disposed in a housing. At least a portion of the working electrode includes rhodium metal. An electrical circuit is disposed in the housing and configured to be electronically coupled to the electrodes. The electrical circuit is operative to: (a) bias the working electrode at a voltage of less than about 0.4 V, and (b) measure a current corresponding to the concentration of the target analyte. A communications module is electrically coupled to the electrical circuit and configured to display a concentration of the target analyte, and/or communicate data between the electrical circuit and an external device. The electrodes are movable between a first configuration in which the electrodes are substantially disposed inside the housing, and a second configuration in which at least a portion of the electrodes is disposed outside the housing.

### CROSS-REFERENCE TO RELATED APPLICATIONS

This application claims priority to U.S. Provisional Application No. 61/951,667 filed Mar. 12, 2014, entitled “Wearable Electrochemical Sensor and Method,” the disclosures of which is hereby incorporated herein by reference in its entirety.

### BACKGROUND

Embodiments described herein relate generally to wearable electrochemical sensors and sensing systems, and in particular to wearable electrochemical sensing systems that include a sensor configured to be inserted into the body of a user.

Electrochemical sensors are defined as sensors that employ an electronic parameter, for example, current, voltage, capacitance, impedance, or any other electronic parameter to measure the concentration of a target analyte, for example, a chemical or biochemical analyte. Among these electrochemical sensors, amperometric electrochemical sensors (i.e., current measuring sensors) are most popular. Amperometric electrochemical sensors can include a working electrode, a reference electrode and optionally, a ground electrode which is electronically coupled via an electrical circuit, for example, a potentiostat. The working electrode is biased at a predetermined positive (i.e., oxidation) or a predetermined negative (i.e., reduction) voltage, capable of oxidizing or reducing the target analyte, respectively. The redox reaction produces a current which is measured and compared with calibration plots to determine the concentration of the target analyte.

Known amperometric electrochemical sensors are also used as biosensors for sensing a non-electroactive target analyte, for example, a biomolecule such as glucose. Such known amperometric electrochemical sensors can include a biosensing molecule such as, for example, an enzyme or a synthetic biocatalyst immobilized on the surface of the working electrode. The biosensing molecule can catalytically decompose the non-electroactive biomolecule to yield an electroactive molecule and a by-product. For example, glucose oxidase decomposes glucose to yield gluconic acid, which is non-electroactive, and hydrogen peroxide which is electroactive. The electroactive hydrogen peroxide is oxidized or reduced on the surface of the working electrode to produce a current which is measured and is correlated to the concentration of the target analyte.

Known amperometric electrochemical sensors are often biased at a relatively high voltage, for example, a voltage of higher than about 0.5 V, to be able to oxidize or reduce the target analyte or its electroactive byproduct. At such high voltages, interfering electroactive chemical species that can also be present in a sample (e.g., a biological sample) that includes the target analyte can also get oxidized or reduced on the working electrode. This can significantly add to the noise and substantially reduce the signal to noise ratio of the electrochemical sensing system. For example, known enzymatic electrochemical sensors for measuring glucose in blood can include a working electrode biased at a voltage of 0.7 V, the optimum voltage for oxidizing hydrogen peroxide, which is the electroactive by-product of the enzymatic reaction. Blood however, can also include ascorbic acid, uric acid, and/or acetaminophen, which can all be oxidized at the 0.7 V voltage and contribute to the noise. Interferent blocking and/or repelling membranes can be used on the surface of the working electrode, however, they can reduce diffusion of the target analyte to the working electrode surface, thereby reducing the electrochemical signal. Known electrochemical systems also include working electrodes that use multi-step redox pathways (e.g., multiple enzymes, redox mediators, etc.) to facilitate electron transfer from or to the target analyte. This can allow biasing of the working electrode but can add to the complexity of the system, increase manufacturing costs, and reduce the signal amplitude.

Electrochemical sensing systems can be used for in vitro sensing i.e., sensing in a non-native environment, for example, outside the body of a user (e.g., sensing in a sample such as a bodily fluid disposed in a test tube or a test container). Electrochemical sensing systems can also be used for in vivo sensing i.e., sensing in a native environment, for example, sensing inside the body of the user. In such instances, the sensors can be inserted inside the body of the user to contact a bodily fluid, for example, blood of the user and sense the target analyte present in the bodily fluid. These sensors generally are inserted into the body of the user every time a sensing measurement is desired. However, repeatable insertion of the sensors into the body of the user can be cumbersome and cause unnecessary pain to the user. Furthermore, for making a plurality of measurements over a period of time, the user might dispose the in vivo electrochemical sensing system on the body of the user every time a measurement is desired. This can create user fatigue where the user forgets to adhere to a measurement schedule or deliberately ignores a measurement schedule, for example, due to laziness.

Thus, it is an enduring goal of electrochemical sensing systems to develop new electrochemical sensing systems that can allow facile insertion of the sensing electrodes into the body of the user and are easy to operate.

### SUMMARY

Embodiments described herein relate generally to wearable electrochemical sensors and sensing systems, and in particular to wearable electrochemical sensing systems that include a sensor configured to be inserted into the body of a user. In some embodiments, an electrochemical sensing system includes a housing configured to be removably associated with a user. The electrochemical sensing system includes a working electrode and a reference electrode, which can at least partially be disposed in the housing. At least a portion of the working electrode includes rhodium metal. An electrical circuit is disposed in the housing and configured to be electronically coupled to the working electrode and the reference electrode. The electrical circuit is operative to: (a) bias the working electrode at a voltage of less than about 0.4 V such that a target analyte decomposes, and (b) measure a current corresponding to the concentration of the target analyte. A communications module is electrically coupled to the electrical circuit and is configured to at least one of display a concentration of the target analyte, and communicate data between the electrical circuit and an external device. The working electrode and the reference electrode are movable between a first configuration in which the working electrode and the reference electrode are substantially disposed inside the housing, and a second configuration in which at least a portion of the working electrode and the reference electrode is disposed outside the housing. In some embodiments, the electrochemical sensing system further includes an insertion mechanism configured to move the working electrode and the reference electrode between the first configuration and the second configuration.

### BRIEF DESCRIPTION OF THE DRAWINGS

FIG. 1 is a schematic illustration of an electrochemical sensing system, according to an embodiment.

FIGS. 2A and 2B are schematic illustrations of an electrochemical sensing system in a first configuration and a second configuration respectively, according to an embodiment.

FIGS. 3A and 3B are schematic illustrations of an electrochemical sensing system in a first configuration and a second configuration respectively, according to an embodiment.

FIG. 4A is an enlarged view of a portion of the electrochemical sensing system of FIG. 3A shown by the arrow 4, which includes a penetration member, according to an embodiment. FIG. 4B is a cross-sectional view of the penetration member of FIG. 4A.

FIG. 5 shows a side view of a penetration member, according to an embodiment.

FIG. 6 shows a side view of a penetration member, according to an embodiment.

FIGS. 7A and 7B are schematic illustrations of an electrochemical sensing system that includes a first penetration member and a second penetration member in a first configuration and a second configuration respectively, according to an embodiment.

FIG. 8 shows a side cross-sectional view of a portion of a penetration member, according to an embodiment

FIGS. 9A and 9B are schematic illustrations of an electrochemical sensing system that includes a biasing member in a first configuration and a second configuration respectively, according to an embodiment.

FIGS. 10A and 10B are schematic illustrations of an electrochemical sensing system in a first configuration and a second configuration respectively, according to an embodiment.

FIG. 11 is a top perspective view of an electrochemical sensing system in a first configuration, according to an embodiment.

FIG. 12 is a top perspective view of the electrochemical sensing system of FIG. 11 in a second configuration.

FIG. 13 is a bottom perspective view of the electrochemical sensing system of FIG. 11 in the second configuration.

FIG. 14 is a top perspective view of an electrochemical sensing system in a first configuration, according to an embodiment.

FIG. 15 is a top perspective view of the electrochemical sensing system of FIG. 14 in a second configuration.

FIG. 16 is a bottom perspective view of the electrochemical sensing system of FIG. 14 in the second configuration.

FIG. 17 is an illustration of a display included in a communications module of an electrochemical sensing system, according to an embodiment.

FIG. 18 is an illustration of a display included in a communications module of an electrochemical sensing system, according to an embodiment.

FIG. 19 is a plot showing the intensity of exercise performed by a user based on the glucose level measured by a wearable electrochemical sensing system.

FIG. 20 is a plot showing GLUCOSE VARIATION INDEX™ of a user over a period of 24 hours.

FIG. 21 is an illustration of a display included in a communications module of an electrochemical sensing system, according to an embodiment.

FIG. 22 is an illustration of a display included in a communications module of an electrochemical sensing system, according to an embodiment.

### DETAILED DESCRIPTION

Electrochemical sensing systems configured to measure the concentration of a target analyte in vivo, for example, in a bodily fluid of a user, can include sensors that can be inserted into the body a user. Such sensors can be inserted into the body to establish contact with the bodily fluid each time a measurement of the analyte concentration is desired. However, repeatable insertion of the sensors into the body of the user can be cumbersome and cause unnecessary pain to the user.

Furthermore, conventional electrochemical systems often include working electrodes that are polarized at a relatively high bias voltage to oxidize or reduce a target analyte and produce a measurable current. Such high bias voltages can also oxidize or reduce interfering species, for example, interfering electroactive species present in a sample that includes the target analyte. This contributes to the “noise” (also referred to as “interference”) in the tested sample and can substantially decrease the signal to noise ratio.

Embodiments described herein relate generally to wearable electrochemical sensors and sensing systems, and in particular to wearable electrochemical sensing systems that include an insertion mechanism. Embodiments of the electrochemical sensing system described herein offer several advantages including, for example: (1) provide facile insertion of the sensors, for example, a working electrode and/or a reference electrode into the body of the user on demand; (2) provide means for inserting the sensors into the body without the need of complex insertion mechanisms; (3) a penetration member that includes the working electrode and the reference electrode, such that the sensing measurement can be performed with a single insertion; and (4) small size, light weight, and ergonomic design, such that electrochemical sensing system is wearable and minimally impacts the comfort of the user.

In some embodiment; an electrochemical sensing system includes a housing configured to be removably associated with a user. The electrochemical sensing system includes a working electrode and a reference electrode, which can be at least partially disposed in the housing. At least a portion of the working electrode includes rhodium metal. An electrical circuit is disposed in the housing and configured to be electronically coupled to the working electrode and the reference electrode. The electrical circuit is operative to: (a) bias the working electrode at a voltage of less than about 0.4 V such that a target analyte decomposes, and (b) measure a current corresponding to the concentration of the target analyte. A communications module is electrically coupled to the electrical circuit. The communications module is configured to at least one of display a concentration of the target analyte, and communicate data between the electrical circuit and an external device. The working electrode and the reference electrode are movable between a first configuration in which the working electrode and the reference electrode are substantially disposed inside the housing, and a second configuration in which at least a portion of the working electrode and the reference electrode is disposed outside the housing. In some embodiments, the electrochemical sensing system further includes an insertion mechanism which is configured to move the working electrode and the reference electrode between the first configuration and the second configuration.

In some embodiments, an electrochemical sensing system includes a working electrode that includes rhodium, and a reference electrode. The electrochemical sensing system also includes a housing which is configured to removably associate the working electrode and the reference electrode with the body of a user. The housing includes a base portion and a movable portion. The housing is movable between an unassembled position and an assembled position by moving the movable portion with reference to the base portion. An electrical circuit is disposed in the base portion of the housing and configured to be electronically coupled to the working electrode and the reference electrode. The electrical circuit is operative to: (a) bias the working electrode at a voltage of less than about 0.4 V such that a target analyte decomposes, and (b) measure a current corresponding to the concentration of the target analyte. A communications module is disposed in the movable portion of the housing and configured to be electrically coupled to the electrical circuit. The working electrode and the reference electrode are movable between a first configuration in which the working electrode and the reference electrode are substantially disposed inside the housing, and a second configuration in which at least a portion of the working electrode and the reference electrode is disposed outside the housing. In some embodiments, the working electrode and the reference electrode are moved from the first configuration to the second configuration by moving the housing between the unassembled position and the assembled position. In some embodiments, the communications module is configured to at least one of a display a concentration of the target analyte, and transfer data between an electrical circuit and an external device.

As used herein, the term “about” and “approximately” generally mean plus or minus 10% of the value stated, e.g., about 250 μm would include 225 μm to 275 μm, about 1,000 μm would include 900 μm to 1,100 μm.

As used herein, the term “target analyte” refers to a chemical or a biochemical that can be sensed by embodiments of the electrochemical sensing system described herein.

As used herein, the term “electroactive” means a chemical or a biochemical that can be electrochemically oxidized or reduced at an electrode biased at an appropriate biasing voltage.

As used herein, the term “interferents” refers to chemicals or biochemicals (except a target analyte) that are electroactive and can undergo a redox reaction at a working electrode included in any embodiments of the electrochemical sensing system described herein, that contributes to noise.

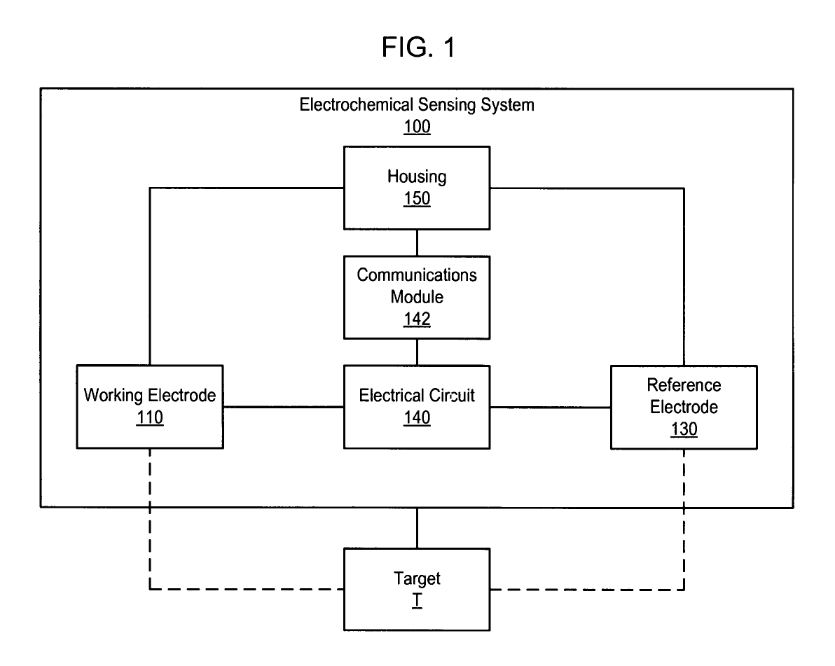

FIG. 1 shows a schematic illustration of an electrochemical sensing system 100, according to an embodiment. The electrochemical sensing system 100 includes a working electrode 110, a reference electrode 130, an electrical circuit 140, a communications module 142, and a housing 150. The electrochemical sensing system 100 is configured to be removably disposed on a target T, for example, skin of a user (e.g., a patient) such that the electrochemical sensing system 100 is wearable. Furthermore, the working electrode 110 and the reference electrode 130 are configured to be moveable between a first configuration wherein the working electrode 110 and the reference electrode 130 are disposed substantially within the housing 150, and a second configuration in which at least a portion of the working electrode 110 and the reference electrode 130 are disposed outside the housing 150, as described herein. The working electrode 110 and the reference electrode 130 can be inserted into the target T, for example into the skin of the target T to contact a bodily fluid, for example blood, and electrochemically decompose a target analyte included in the bodily fluid to measure the target analyte concentration.

The working electrode 110 can include a rhodium electrode, or an electrode having rhodium disposed thereon. The working electrode 110 can be configured to oxidize a target analyte at a biasing voltage of less than about 0.4 V such that oxidation and reduction of one or more interfering species on the working electrode 110 is substantially reduced. Without being bound by any particular theory, rhodium can catalytically oxidize or reduce the target analyte, such that a relatively low bias voltage, for example, less than about 0.4 V, is sufficient to oxidize or reduce the target analyte or an electroactive by-product of the target analyte. In some embodiments, the biasing voltage can be, less than about 0.35 V, less than about 0.3 V, less than about 0.25 V, less than about 0.20 V, less than about 0.15 V, less than about 0.1 V, less than about 0.05 V, or about 0 V, inclusive of all ranges therebetween. The bias voltage can be sufficiently low such that interferents (e.g., interfering electroactive species present in a sample that includes the target analyte) are not oxidized or reduced on the working electrode 110. In some embodiments, the working electrode 110 can be formed from an oxide of rhodium, for example, RhO₂, Rh(OH)₃, or Rh₂O₃. In some embodiments, a blend of rhodium and another metal, for example, ruthenium, platinum, palladium, gold, nickel, any other suitable metal or alloy, can be used to form the working electrode 110.

In some embodiments, the working electrode 110 can be a pure rhodium electrode. In some embodiments, the working electrode 110 can include a substrate on which rhodium is disposed. The substrate can be formed from any suitable conductive material that has good adhesion with rhodium, for example, chromium, titanium, nitinol, gold, platinum, nickel, palladium, stainless steel, any other suitable material or combination thereof. The rhodium can be disposed on the substrate using any suitable process. For example, in some embodiments, the rhodium can be electroplated over the substrate. Any suitable rhodium salt solution can be used to electroplate rhodium on the substrate, for example, a rhodium sulfate solution, a rhodium chloride solution, any other rhodium plating solution or combination thereof. The deposition voltage and/or time can be controlled to obtain a predetermined thickness of rhodium on the substrate.

In some embodiments, the rhodium can be disposed on the substrate using a co-extrusion process, for example, when forming a cylindrical working electrode 110. In some embodiments, a physical deposition process can be used to dispose rhodium on the substrate. Such processes can include, for example, casting or a physical vapor deposition (PVD) process such as, for example, e-beam evaporation, thermal evaporation, sputtering, atomic layer deposition (ALD), pulsed laser deposition (PLD), ion implantation, any other physical vapor deposition process or a combination thereof. In some embodiments, a chemical vapor deposition (CVD) process can be used to dispose rhodium on the substrate. Suitable processes can include, for example, low pressure chemical vapor deposition (LPCVD), plasma enhanced chemical vapor deposition (PECVD), molecular beam epitaxy (MBE), any other suitable chemical vapor deposition process or combination thereof. In some embodiments, self assembly can be used to dispose rhodium on the substrate. For example, rhodium nanoparticles can be urged to self assemble on the substrate to form the working electrode 110. Use of a substrate formed from a material which has a high adhesion with rhodium and/or rhodium oxide can enable the deposition of the rhodium (or rhodium electrode) on any suitable substrate, for example, a plastic substrate (e.g., a high density polyethylene (HDPE) or a poly tetrafluoroethylene (PTFE)) substrate, a silicon substrate, or a TEFLON® substrate.

The working electrode 110 can have any suitable shape or size. For example, in some embodiments, the working electrode 110 can be a rod having a circular, oval, or polygonal cross-section. In such embodiments, the working electrode 110 can be a solid cylindrical electrode or a hollow cylindrical electrode (e.g., a cylindrical electrode that defines a lumen). In some embodiments, the working electrode 110 can be a needle type electrode which can, for example, be configured to be inserted into an animal or human body for measuring the concentration of the target analyte. In some embodiments, the working electrode 110 can be a flat electrode, for example, a flat plate, a disc, a solid state microfabricated electrode (e.g., of the type used in MEMS devices), or a screen printed electrode. In some embodiments, at least a portion of the working electrode 110 can be insulated with an insulating material, for example, rubber, TEFLON®, plastic, parylene, silicon dioxide, silicon nitride, any other suitable insulation material or combination thereof. The insulation material can, for example, be used to define an active area of the working electrode 110.

In some embodiments, the working electrode 110 can be subjected to a surface modification process to modify a surface area of the rhodium, for example, to provide a larger surface area for the target analyte to undergo the redox reaction at the working electrode 110. Such surface modification processes can include, for example, etching (e.g., etching in an acidic or basic solution), voltage cycling (e.g., cyclic voltammetry), electrodeposition of colloidal rhodium, electrospraying, any other suitable surface modification process or combination thereof. In some embodiments, the rhodium metal can be oxidized to yield a rhodium oxide (e.g., rhodium dioxide) layer on the substrate. For example, the working electrode 110 can be immersed in an acidic bath, exposed to an oxygen plasma, corona discharge, any other suitable process or combination thereof can be used to oxidize the rhodium disposed on the substrate of the working electrode 110.

In some embodiments, a biosensing molecule (not shown) can optionally be disposed on the working electrode 110. In such embodiments, the target analyte can be a biomolecule which is non-electroactive. Such target analytes can include, for example, glucose, sucrose, glutamate, lactate, cholesterol, alcohol, aspartate transaminase, alkaline transaminase, alkaline phosphatase, urea, ascorbate, pyruvate, L-arginine, creatine, choline, or any other biomolecule. The biosensing molecule can be configured to catalytically decompose the non-electroactive target analyte and yield an electroactive by-product. The electroactive by-product can thereby, be oxidized or reduced at the working electrode 110 to yield a current which corresponds to the concentration of the target analyte. In some embodiments, the biosensing molecule can be an enzyme such as, for example, glucose oxidase, glutamate oxidase, lactate oxidase, lactate dehydrogenase, cholesterol oxidase, ascorbate oxidase, pyruvate oxidase, myokinase, arginase, choline oxidase, creatine phosphokinase, phosphatase, any other suitable enzyme or combination thereof. For example, glucose oxidase can be disposed on the working electrode 110 for measuring the concentration of glucose (i.e., the target analyte) which is non-electroactive. The glucose oxidase enzymatically decomposes the glucose to yield gluconic acid and hydrogen peroxide. The hydrogen peroxide can be oxidized on the working electrode 110 at the low bias voltage of less than about 0.4 V (e.g., about 0.3 V) to produce a current, which can be correlated to the concentration of the glucose.

In some embodiments, a single biosensing molecule can be used to decompose the non-electroactive target analyte to yield an electroactive by-product. In some embodiments, a series of biosensing molecules can be disposed on the working electrode 110. For example, a first biosensing molecule can decompose the non-electroactive target analyte into intermediate non-electroactive by-products. A second biosensing molecule can then decompose at least one of the intermediate non-electroactive by-products to yield a final electroactive by-product. In some embodiments, a mediator or a transducer can be included with the biosensing molecule. The mediator or the transducer can serve as an intermediate electron carrier which can facilitate electron transfer to or from the working electrode 110, thereby reducing the bias voltage required to perform the redox reaction.

In some embodiments, the electrochemical sensing system 100 can include a plurality of working electrodes 110, for example, 2, 3, 4 or even more working electrodes. Each of the working electrodes 110 can be paired with the reference electrode 130. In some embodiments, each of the plurality of working electrodes can be substantially similar to each other and configured to measure the same target analyte. In this manner, each working electrode can provide redundancy such that if one working electrode malfunctions, the target analyte concentration can still be measured by the other working electrodes. Furthermore, in such embodiments, the signal from each electrode can be averaged to record a final measurement. This can, for example, allow repeatable measurements to be made with high confidence. In addition, having 2 separate systems measuring the same analyte can eliminate the requirement for calibration since the system is mathematically overprescribed. In some embodiments, each working electrode 110 can include a different biomolecule 120 disposed on the working electrode 110. For example, a first working electrode 110 can include glucose oxidase for measuring glucose and a second working electrode 110 can include a lactate dehydrogenase, for measuring lactate. In this manner, the electrochemical sensing system 100 can be used as a multi analyte sensor.

In some embodiments, the electrochemical sensing system 100 can include a plurality of working electrodes 110 configured to reduce noise. For example, the electrochemical sensing system 100 can include a first working electrode and a second working electrode. The first working electrode and the second working electrode can be substantially similar to the working electrode 110 or any other working electrode described herein. In some embodiments, the first working electrode can include a biosensing molecule 120 such as, for example, glucose oxidase disposed thereon. The second working electrode can be substantially similar to the first working electrode except that the biosensing molecule 120 is not disposed on the second working electrode. Each of the first working electrode and the second working electrode can be paired with the reference electrode 130 and biased at the same voltage, for example, about +0.4 volts. In such embodiments, the first working electrode will measure a first signal that includes the redox current because of the oxidation or reduction of a by-product of the target analyte as well as a noise (e.g., electromagnetic noise, or noise due to any side reactions such as, for example, redox reactions of interferents). In contrast, the second working electrode will measure a second signal (i.e., current) that corresponds to the noise only. Without being bound by any particular theory, electrodes that are substantially similar to each other will measure the same noise. Therefore the noise measured by the second working electrode can be substantially similar to the noise measured by the first working electrode. Thus, the second signal can be subtracted from the first signal to remove the noise measured by the first working electrode. In this manner, a substantially noise free signal corresponding to the electrochemical redox reaction of the target analyte can be measured by the electrochemical sensing system 100.

In some embodiments, the electrochemical sensing system 100 can include a plurality of working electrodes (e.g., two, three, four, or even more) which are biased at different biasing potentials and configured to eliminate calibration. For example, the electrochemical sensing system 100 can include a first working electrode and a second working electrode. Each of the first working electrode and the second working electrode can be substantially similar to the working electrode 110 or any other working electrode described herein. Furthermore, the first working electrode and the second working electrode can be substantially similar to each other. The first working electrode can be biased at a first voltage, for example, about +0.4 volts. The second working electrode can be biased at a second voltage greater than the first voltage, for example, about +0.7 volts. Without being bound by any particular theory, the first working electrode can measure a first signal that includes the redox current of the target analyte, and has a first magnitude. The second working electrode can measure a second signal that also includes the redox current of the target analyte. However because the second voltage is higher than the first voltage, the magnitude of the redox signal measured by the second electrode can be higher than the magnitude of the redox signal measured by the first electrode, such that the second signal has a second magnitude higher than the first magnitude. Since the first working electrode and the second working electrodes are substantially similar to each other, the signals measured by each electrode can be ratiometrically related to each other. Thus, the first signal can be combined with the second signal to eliminate the need for calibration since both are solving the same mathematical equation.

For example, a calibration plot of an electrochemical sensor is generally a straight line that can be described by the calibration equation;

Y=mX+b (1)

where X is the concentration of the target analyte, Y is the current, m is the slope of the line and b is the y-axis intercept. The first electrode can have a first calibration equation

Y₁=m₁X+b₁ (2)

and the second electrode can have a second calibration equation

Y₂=m₂X+b₂ (3)

Without being bound by any particular theory, since Y₁, Y₂ and b₁, b₂ can be correlated based on the predetermined biased voltage by a simple ratio, this can eliminate the need for external calibration.

In some embodiments, the biosensing molecule 120 can be a synthetic redox-active receptor, for example, a viologen or a conjugated pyridinium. The synthetic redox-active receptor can be configured to be moved between different electronic states. For example, the synthetic redox-active receptor can bind or otherwise interact with the target analyte TA to change the synthetic receptor's reduction potential. The binding or otherwise interaction of the synthetic redox-active receptor with the target analyte TA can be an equilibrium reaction in which the target analyte TA does not decompose. The synthetic redox-active receptor can then communicate the accepted electron to the working electrode 110 and move into a different electronic state. This generates a current that can be measured by the electrical circuit 140, as described herein. In this manner a synthetic redox-active receptor can be used to electrochemically sense the target analyte TA, without the target analyte TA being consumed. Such synthetic redox-active receptors can have a higher stability than biomolecules. Thus, they can allow for better stability, lesser drift and longer lifetime of the working electrode 110.

The biosensing molecule can be disposed on the surface of the working electrode 110 using any suitable means. In some embodiments, the biosensing molecule can be physically adsorbed on the surface. In some embodiments, the biosensing molecule can be adsorbed to the surface of the rhodium on the working electrode 110, for example, using thiol chemistry. In some embodiments, the biosensing molecule can be covalently coupled to the surface of the rhodium on the working electrode 110, for example, using silane chemistry. In some embodiments, the biosensing molecule can be ionically bonded to the surface of the rhodium on the working electrode 110, for example, using oppositely charged surface ions. In some embodiments, the biosensing molecule can be suspended in a porous membrane, for example, a polyurethane membrane, a glutaraldehyde membrane, a silicone membrane, a sol-gel membrane, a NAFION® membrane, plasma deposited polyethylene oxides, a hydrogel membrane, or any other suitable membrane or combination thereof, which is disposed on an outer surface of the working electrode 110.

In some embodiments, a selectivity layer (not shown) can be disposed on the working electrode 110, for example, disposed between the surface of the rhodium and the biosensing molecule. The selectivity layer can be configured to prevent electroactive interferents from coming in contact with the working electrode 110 and undergoing a redox reaction. For example, in some embodiments, the selectivity layer can be configured to repel oppositely charge ionic interferents. For example, a NAFION® selectivity layer can be disposed between an outer surface of the working electrode 110 and the biosensing molecule 120. The NAFION® is inherently negatively charged and repels negatively charged interferents such as, for example, ascorbic acid, but allows a neutral target analyte, such as hydrogen peroxide to diffuse through the NAFION® to the working electrode 110. In some embodiments, the selectivity layer can be a size exclusion layer, for example, a cellulose acetate layer. Such a selectivity layer can be porous and define a pore size such that only the small target analyte, for example, hydrogen peroxide can diffuse through the pores of the selectivity layer, while larger interferents such as, for example, ascorbic acid are blocked.

In some embodiments, a porous membrane (not shown) can be disposed over the biosensing molecule. The porous membrane can ensure substantially stable diffusion of the target analyte to the biosensing molecule over the operational lifetime of the working electrode 110. Stable diffusion can ensure that any changes in the amperometric current measured by the electrochemical sensing system 100 is substantially due to a change in concentration of the target analyte and is not due to a variable flux of the target analyte to the biosensing molecule. In some embodiments, the porous membrane can be biocompatible. In some embodiments, the porous membrane can also prevent fouling of the working electrode 110, for example, biofouling due to proteins present in a biological sample. In some embodiments, the porous membrane can include antioxidants such as catalase to protect the biosensing molecule from reactive oxygen species. Examples of materials which can be used to form the porous membrane can include, for example, polyurethane, silicone, glutaraldehyde, a sol-gel, silicones, hydrogels, plasma-deposited polyethylene oxides and any other suitable diffusivity layer or combination thereof.

The reference electrode 130 is electronically coupled to the working electrode 110 via the electrical circuit 140. The reference electrode 130 can include any suitable reference electrode that can provide a stable reference voltage for the working electrode 110 and does not get consumed by the oxidation or reduction reaction, thereby providing longer shelf life, no usage limitations due to reference consumption, and substantially reduce signal drift. Suitable materials for the reference electrode 130 can include, for example, metal oxides (e.g., iridium oxide, ruthenium oxide, platinum oxide, palladium oxide, rhodium oxide, etc.), metal halides, conducting polymers (e.g., polyethylene dioxythiophene:polystyrene sulfonate (PEDOT:PSS), any other suitable stable reference electrode or combination thereof. In some embodiments, the reference electrode 130 can include rhodium and its oxides (e.g., RhO₂, Rh(OH)₃, Rh₂O₃, etc.). In some embodiments, the reference electrode 130 can include iridium and its oxides. In some embodiments, the reference electrode 130 can include palladium and its oxides.

The reference electrode 130 can have any shape or size. For example, in some embodiments, the reference electrode 130 can be a rod having a circular, oval, or polygonal cross-section. In some embodiments, the reference electrode 130 can be a needle type electrode which can, for example, be configured to be inserted into an animal or human body along with the working electrode 110. In some embodiments, the reference electrode 130 can be a flat electrode, for example, a flat plate, a disc, a solid state microfabricated electrode (e.g., of the type used in MEMS devices), or a screen printed electrode. In some embodiments, at least a portion of the reference electrode 130 can be insulated with an insulating material, for example, rubber, TEFLON®, plastic, polyimide, parylene, silicone, silicon oxide, silicon nitride, any other suitable insulation material or combination thereof. The insulation material can, for example, be used to define an active area of the reference electrode 130. In some embodiments, the reference electrode 130 can have the same shape as the working electrode 110.

In some embodiments, a porous membrane (not shown) can be disposed over the surface of the reference electrode 130, for example, to prevent fouling of the reference electrode. For example, proteins in a biological solution (e.g., blood) in which the target analyte is being detected, can adhere to the surface of the reference electrode, thereby fouling the reference electrode 130, which can cause the formal voltage of the reference electrode 130 to drift. The porous membrane, for example, a biocompatible porous membrane can prevent the proteins from adhering to the reference electrode, thereby reducing fouling. Examples of materials which can be used to form the porous membrane can include, for example, polyurethane, glutaraldehyde, a sol-gel, silicones, hydrogels, plasma deposited polyethylene oxides, any other suitable diffusivity layer or combination thereof.

As shown in FIG. 1, the electrochemical sensing system 100 includes the working electrode 110 and the reference electrode 130 such that the electrochemical sensing system 100 is configured to operate in a two pole sensor system. The reference electrode 130 functions thereby as a pseudo-reference electrode, that is provides a reference voltage for the working electrode 110 to be biased against, as well as communicates electrons to or from the sample (e.g., a liquid or gaseous sample) that includes the target analyte. Conventional pseudo-reference electrodes such as, for example, silver/silver chloride (Ag/AgCl) electrodes, generally do not maintain a constant reference voltage. Instead the reference can vary predictably with external conditions, for example, the pH and temperature of the electrolyte can affect the reference potential. If the conditions are known, the potential can be calculated and the electrode can be used as a reference, however, in many cases a calibration measurement has to be performed for reliable measurements. Many conventional pseudo-reference electrodes, for example, Ag/AgCl electrodes, can only operate in limited pH or temperature ranges. Furthermore, many conventional pseudo-reference electrodes (e.g., the Ag/AgCl reference electrode) can be consumed during the electrochemical reaction, because, the pseudo-reference electrode not only provides a reference voltage for the working electrode, but also communicate electrons to or from the sample. This can lead to oxidation/reduction of the reference electrode material which can eventually get consumed. Thus, conventional reference electrodes generally have a short life.

In contrast, the pseudo-reference electrode 130 described herein, which can be formed from a metal oxide or metal/metal oxide (e.g., oxides of rhodium) can be insensitive to the ambient conditions such as, for example, the pH and temperature, and therefore provide a stable reference voltage. The reference electrode 130 can also be relatively inert such that the reference electrode 130 is not consumed and has a relatively long life. Moreover, the reference electrode 130 can also reduce and/or provide more efficient electron transfer thereby substantially increasing the sensitivity of the electrochemical measurement as compared to a conventional reference electrode (e.g., a Ag/AgCl reference electrode)

In some embodiments, the electrochemical sensing system 100 can further include a third counter electrode (not shown). In such embodiments, the electrochemical sensing system 100 can be operated in a three electrode configuration such that the electrons are communicated to or from the sample via the counter electrode. In such embodiments, the reference electrode 130 only serves to provide an electronic reference for the working electrode 110.

In some embodiments, the electrochemical sensing system 100 can include a penetration member (not shown). The penetration member can, for example, be a needle or a pointed member having a sharp tip such that the penetration member can be inserted into the target T, for example, penetrate the skin and contact a bodily fluid. The working electrode 110 can be disposed on a first surface and the reference electrode 130 can be disposed on a second surface of the penetration member. In this manner, the working electrode 110 and the reference electrode 130 can be inserted into the target T along with the penetration member and contact the bodily fluid to electrochemically decompose the target analyte. In some embodiments, the penetration member can include a cylindrical member (e.g., having a circular cross-section) that includes a sharp tip and defines a lumen. For example, the penetration member can include a hollow needle (e.g., a 30 gage needle). The working electrode 110 can be disposed on a first surface of the penetration member, for example, an inside surface that defines the lumen. Moreover, the reference electrode 130 can be disposed on a second surface of the penetration member, for example, an outer surface of the penetration member. In such embodiments, the penetration member can be formed from an insulating material (e.g., plastic). In some embodiments, the penetration member can be formed from an electrically conducting material, for example, metal or metal alloys. In such embodiments, an insulating layer can be disposed between the working electrode 110 and the reference electrode 130. The penetration member can also include one or more apertures on a side wall of the penetration member. The aperture can be configured to allow the bodily fluid to flow inside the lumen of the penetration member, for example, by allowing air to escape from the lumen. This can establish fluidic contact between the working electrode 110 and the reference electrode 130 via the bodily fluid thereby enabling electrochemical decomposition of the target analyte on the working electrode 110.

In some embodiments, the penetration member can be configured such that the working electrode 110 is disposed on a first portion of a first surface, and the reference electrode 130 is disposed on a second portion of the first surface of the penetration member. For example, the working electrode 110 can be disposed on a first half of an outer surface of the penetration member, and the reference electrode 130 can be disposed on a second half of the outer surface of the penetration member. In such embodiments, the penetration member can be a solid member which does not define a lumen.

The electrical circuit 140 is configured to bias the working electrode 110 at a predetermined operating voltage, for example, a voltage of less than about +0.4V and measure a redox current due to the oxidation or reduction of the target analyte or an electroactive by-product of the target analyte. In some embodiments, the electrical circuit 140 can include a transimpedance amplifier circuit configured to convert current to an amplified voltage. In some embodiments, the electrical circuit 140 can include an analog to digital converter configured to digitize the input current measurement. For example, the electrical circuit 140 can include a differential analog to digital converter which can increase noise rejection in the voltage measurement. The bias voltage can be communicated into a low end differential input of the analog to digital converter configured to provide a pseudo-negative range. This can, for example, allow digital filtering to remain accurate when noise remains in the low measurement range (e.g., to enhance the limit of detection). In some embodiments, the electrical circuit 140 can include operational amplifiers configured to amplify the measured signal. In some embodiments, the electrical circuit 140 can include a filtering circuit, for example, a low pass filter, a high pass filter, a band pass filter, any other suitable filtering circuit, or combination thereof, configured to substantially reduce signal noise. In some embodiments, the electrical circuit 140 can include a potentiostat circuit, for example, a programmable potentiostat circuit, configured to bias the working electrode 110 at the predetermined voltage. For example, the potentiostat circuit can be configured to bias the working electrode 110 at a biasing voltage in the range of about −0.6 V to about +0.5 V, for example, about −0.5 V, −0.4 V, −0.3 V, −0.2 V, −0.1 V, 0 V, +0.1 V, +0.2 V, +0.3 V, or about +0.4 V, inclusive of all ranges therebetween.

In some embodiments, the electrical circuit 140 can include a processor, e.g., a microcontroller, a microprocessor, an ASIC chip, an ARM chip, or a programmable logic controller (PLC). The processor can include signal processing algorithms, for example, band pass filters, low pass filters, any other signal processing algorithms or combination thereof. In some embodiments, the processor can be configured to control the bias voltage in real time, for example, to control one or more parameters of the redox reaction in real time. Such parameters can include, for example, electrochemical reaction rate and dynamic range which can be used to reverse or minimize the effects of electrochemical fouling and/or facilitate real time calibration. In some embodiments, the electrical circuit 140 can include a memory configured to store at least one of a redox current data, bias voltage data, user log, or any other information related to the electrochemical reaction. In some embodiments, the memory can also be configured to store a reference signature, for example, a calibration equation. In such embodiments, the processor can be configured to correlate the measured signal (e.g., the redox current) with the reference signature to determine the concentration of the target analyte.

In some embodiments, the electrochemical sensing system 100 can include a communications module 142 electrically coupled to the electrical circuit 140. The communications module 142 can be configured to allow two-way communication with a remote device, for example, a smart phone app, a local computer and/or a remote server. For example, the communications module 142 can communicate the target analyte concentration data from the electrical circuit 140 to the external device. In some embodiments, the communications module 142 can include a communication interface to provide wired communication with the external device, for example, a USB or FireWire interface. In some embodiments, the communications module 142 can include means for wireless communication with the external device, for example, Wi-Fi, Bluetooth®, ANT+, low powered Bluetooth®, Zigbee and the like. In some embodiments, the communications module 142 can include a RFID chip configured to store information, for example, the reference signature or sensing history, and allow a near field communication (NFC) device to read the stored information and/or update the stored information. In some embodiments, the electrochemical sensing system 100 can include a power source, for example, a rechargeable battery, configured to power the electrical circuit 140, the communications module 142 or any other electronic component included in the electrochemical sensing system 100.

In some embodiments, the communications module 142 can include a display configured to display a concentration of the analyte. In some embodiments, the display can be configured to communicate further information to the user, for example, history of use, remaining battery life, wireless connectivity status, and/or visual reminders. In some embodiments, the display can be configured to resemble a dial gage that can include a movable indicator (e.g., a digital needle) and static color coded regions. For example, the color coded regions can include red, green, yellow, or more color coded regions indicating different zones corresponding to the concentration, band, and/or range of the target analyte. For example, in some embodiments, the target analyte can be glucose and the color coded zones can indicate regions of hypoglycemia, hyperglycemia, or euglycemia to the user. Furthermore, the displacement speed of the indicator can be used to indicate the rate of change of the concentration of the target analyte (e.g., glucose). For example, fast or slow ghosting (i.e., movement) of the indicator towards a first side of the dial gage display can indicate that the concentration of the target analyte is rising fast or slow, respectively. Similarly, fast or slow ghosting (i.e., movement) of the indicator towards a second side of the dial gage display can indicate that the concentration of the target analyte is falling fast or slow, respectively. No movement of the indicator can correspond to a stable concentration of the target analyte.

In some embodiments, the display can include a wheel type display (e.g., a digital wheel display). For example, the display can include a movable wheel that includes a color coded regions and a static indicator (e.g., a needle). The color coded regions can include red, green, yellow, or more color coded regions indicating different zones corresponding to the concentration, bands, or ranges of the analyte. For example, in some embodiments, the target analyte can be glucose and the color coded zones can indicate regions of hypoglycemia, hyperglycemia, or euglycemia to the user. Furthermore, the displacement speed of the wheel can be used to indicate the rate of change of the concentration of the target analyte (e.g., glucose). For example, fast or slow rotation of the display in a first direction can indicate that the concentration of the target analyte is rising fast or slow, respectively. Similarly, fast or slow rotation of the wheel in a second direction (e.g., counter clockwise direction) can indicate that the concentration of the target analyte is falling fast or slow, respectively. No movement of the display wheel can correspond to a stable concentration of the target analyte. In some embodiments, the wheel type display can be configured to include one or more shaded zones above or below the indicator which can be configured to indicate a rate of change of glucose.

In some embodiments, the communications module 142 can also include microphones and/or vibration mechanisms to convey audio and tactile alerts. In some embodiments, the communications module 142 can include a means for user input, for example, a button, a switch, and/or a touch screen, to provide an interface for input of at least one of power ON/OFF the electrochemical sensing system 100, reset the electrochemical sensing system 100, trigger communication between the electrochemical sensing system 100 and an external device (e.g., a smart phone). Furthermore, the input interface can also be used to change the display between a visual display (e.g., the dial gage and/or the wheel type display) and a numerical display.

The various components of the electrochemical sensing system 100 are disposed in the housing 150 which is configured to be removably associated to the target T (e.g., the skin of a user). The housing 150 can define an internal volume configured to house the components of the electrochemical sensing system 100. The various components of the electrochemical sensing system 100 can be fixedly or releasably coupled to the housing 150. A releasable adhesive can at least partially coat an underside of the housing 150, for example, to adhere the electrochemical sensing system 100 to the target T, for example, the skin of a patient. The adhesive can be non-toxic, biocompatible, and releasable from human skin. To protect the adhesive until the electrochemical sensing system 100 is ready for use, a removable protective covering can cover the adhesive, in which case the covering can be removed before the housing 150 is applied to the skin. Alternatively, the adhesive can be heat or pressure sensitive, in which case the adhesive can be activated once the device is applied to the skin. Example adhesives include, but are not limited to, acrylate based medical adhesives of the type commonly used to affix medical devices such as bandages to skin. However, the adhesive is not necessary, and may be omitted, in which case the housing can be associated with the skin, or generally with the body, in any other manner. For example, a strap or band can be used.

The housing 150 can be formed from a material that is relatively lightweight and flexible, yet sturdy. The housing 150 also can be formed from a combination of materials such as to provide specific portions that are rigid and specific portions that are flexible. Example materials include plastic and rubber materials, such as polystyrene, polybutene, carbonate, urethane rubbers, butene rubbers, silicone, and other comparable materials and mixtures thereof, or a combination of these materials or any other suitable material can be used. The housing 150 can have a relatively smooth surface, curved sides, and/or otherwise an ergonomic shape. For example, one or more portions of the housing 150 can have dome shape or a tear drop shape, such that the housing 150 is easy to handle and can be concealed within the clothing of the user.

In some embodiments, the housing 150 can include a deformable portion configured to be engaged by a user. For example, the housing 150 can include a base portion which is rigid, and the deformable portion can be a roof, a top portion, or a lid of the housing 150. The deformable portion can be movable between an undeformed position in which the working electrode 110 and the reference electrode 130 are in the first configuration (i.e., substantially disposed within the internal volume defined by the housing), and a deformed position in which the deformable portion urges the working electrode 110 and the reference electrode 130 into the second configuration (i.e., at least a portion of the working electrode 110 and the reference electrode 130 are disposed outside the housing). For example, a user can manually deform the deformable portion. In some embodiment, an actuation mechanism, for example, a piston, a cam, a spring, a push rod, liquid pressure, gas pressure, a piezoelectric actuator, an electrochemical actuator, any other actuation mechanism, or combination thereof can be used to deform the deformable portion. In some embodiments, the deformable portion and/or the base portion can include a locking mechanism, for example, a lock, latch, notches, grooves, indents, detents, friction-fit or snap-fit mechanism, that can maintain the deformable portion in the deformed position. Furthermore, the alarms or otherwise indicators (e.g., audible, visual, or tactile indicators) can be included in the electrochemical sensing system which can be configured to inform a user that the deformable portion is completely deformed.

In some embodiments, the housing 150 can include a retraction member, for example, a spring (e.g., compression, tension, torsion, Belleville, leaf spring, etc.), piston, cam, mechanical linkage, or any other retraction member, which can be configured to urge the deformable portion from the deformed position into the undeformed position.

In some embodiments, the housing 150 can include an insertion mechanism (not shown) configured to move the working electrode 110 and the reference electrode 130 between the first configuration and the second configuration. For example, in some embodiments, the insertion mechanism can include a lumen defined in the housing. One or more components of the electrochemical sensing system 100, for example, the working electrode 110, the reference electrode 130, the electrical circuit 140, and/or the communications module 140 can be slidably disposed in the lumen. Application of a force on the one or more components, for example, by urging the deformable portion into a deformed position can displace the one or more components within the lumen. The working electrode 110 and the reference electrode can be in the first configuration, such that application of the force can urge the working electrode 110 and the reference electrode 130 through one or more apertures defined in the housing 150, and into the second configuration, for example, inserted into the target T. In some embodiments, the communications module 142 can be removably disposed in the lumen and configured to urge the working electrode 110 and the reference electrode 130 into the second configuration. For example, the working electrode 110 and the reference electrode 130 can be in the first configuration, and the communications module 142 can be disposed outside the housing 150. The communications module 142 can be inserted into the lumen such that insertion of the communications module 142 into the lumen urges the working electrode 110 and the reference electrode 130 into the second configuration. In such embodiments, the communications module 142 can be snap-fit or friction fit into the lumen.

In some embodiments, the insertion mechanism can be automated. For example, the insertion mechanism can include a button, a latch, a pull-tab, a push-tab, electrical switch, or any other activation mechanism to activate the insertion mechanism. In some embodiments, the insertion mechanism can also include an actuation mechanism, for example, a piston driven by an electric motor, a mechanical and/or kinematic roller, a piezoelectric actuator, a spring, a gas pressure chamber, a liquid pressure chamber, a cam, any other suitable device or combination thereof. In such embodiment, the actuation mechanism can be configured to engage one or more components of the electrochemical sensing system 100 to move the working electrode 110 and the reference electrode 130 between the first configuration and the second configuration.

In some embodiments, the housing 150 can include a base portion and a movable portion. The housing 150 can be movable between an unassembled position and an assembled position by moving the movable portion with reference to the base portion. For example, in some embodiments, the movable portion can be pivotally mounted to the base portion, for example, by hinges. In such embodiments, the movable portion can rotate about the pivot mount between the unassembled and the assembled position. In some embodiments, the movable portion can be slidably disposed on the base portion. In some embodiments, the housing 150 can be configured such that moving the movable portion from the unassembled position into the assembled position urges the working electrode 110 and the reference electrode 130 from the first configuration into the second configuration. The electrical circuit 140 can be disposed in the base portion of the housing 150 and can, for example, be fixedly coupled to the base portion. The communications module 142 can be disposed in the movable portion of the housing 150 and can, for example, be releasably coupled to the movable portion.

In use, the electrochemical sensing system 100 can be disposed on the target T (e.g., in contact with the skin of a user). The insertion mechanism can be activated using any of the methods described herein, to insert the working electrode 110 and the reference electrode 130 into the target T, such that the working electrode 110 and the reference electrode 130 are in fluidic contact with a bodily fluid of the target T (e.g., blood or interstitial fluid). The electrical circuit 140 can bias the working electrode at a voltage of less than about 0.4 V, to electrochemically decompose a target analyte, or an electroactive by-product of the target analyte, as described herein. The redox current is measured by the electrical circuit 140 and correlated to the concentration of the target analyte. The working electrode 110 and the reference electrode 130 can remain inserted into the target T, or retracted into the housing 150, after performing the electrochemical sensing. The communications module 142 can then show the measured concentration, a rate of change of concentration of the target analyte, and/or physiological state of the target T based on the target analyte concentration. The communications module 142 can also communicate the concentration data to an external device, for example, a smart phone app, a local compute or a remote server. In some embodiments, the electrochemical sensing system 100 can remain disposed on the target T and perform real time electrochemical measurements for extended periods of time, for example, 2 weeks or more. In some embodiments, the electrochemical sensing system 100 can be single use and be disposed after making single measurement. In some embodiments, portions of the electrochemical sensing system can disposable. For example, the working electrode 110 and the reference electrode 130 can be uncoupled from the electrochemical sensing system 100 and be replaced with a fresh set of electrodes after a predetermined number of time and/or sensing measurements.

Having described above various general principles, several embodiments of these concepts are now described. These embodiments are only examples, and many other configurations an electrochemical sensing system are contemplated.

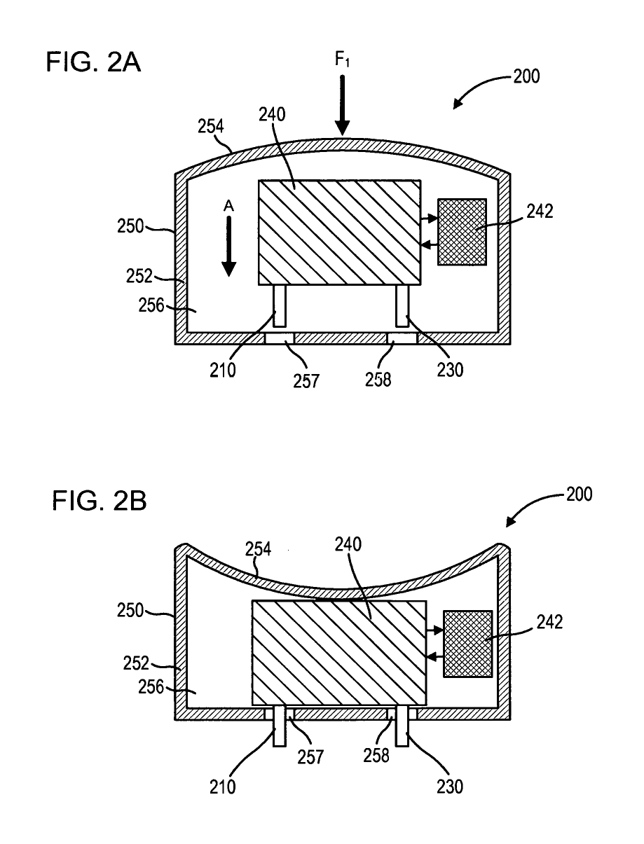

In some embodiments, an electrochemical sensing system can include a housing that has a deformable portion. Referring now to FIGS. 2A and 2B, an electrochemical sensing system 200 includes a working electrode 210, a reference electrode, 230, an electrical circuit 240, a communications module 242, and a housing 250. The electrochemical sensing system 200 can be configured to be associated with a target, for example, the skin of the user, such that the electrochemical sensing system 200 is wearable.

The working electrode 210 can include a rhodium electrode, or an electrode having rhodium disposed thereon. The working electrode 210 can be configured to oxidize a target analyte at a biasing voltage of less than about 0.4 V such that oxidation and reduction of one or more interfering species on the working electrode 210 is substantially reduced. The working electrode 210 can be substantially similar to the working electrode 110 described with respect to the electrochemical sensing system 100, and is therefore not described in further detail herein. In some embodiments, a biosensing molecule can be disposed on the working electrode 210. The biosensing molecule can be operative to decompose a non-electroactive target analyte and yield an electroactive by-product. In some embodiments, the biosensing molecule can include a synthetic redox-active receptor. The biosensing molecule can be substantially similar to the biosensing molecule included in the electrochemical sensing system 100, and is therefore not described in further detail herein.

The reference electrode 230 is electronically coupled to the working electrode 210 via the electrical circuit 240. The reference electrode 230 can include any suitable reference electrode that can provide a stable reference voltage for the working electrode 210 and does not get consumed by the oxidation or reduction reaction, thereby providing longer shelf life, no usage limitations due to reference consumption, and substantially reduce signal drift. For example, the reference electrode 230 can include rhodium and its oxides, iridium and its oxides or palladium and its oxides. The reference electrode 230 can be substantially similar to the reference electrode 130 included in the electrochemical sensing system 100, and is therefore not described in further detail herein.

The electrical circuit 240 is configured to bias the working electrode 210 at a predetermined operating voltage, for example, a voltage of less than about 0.4 V and measure a redox current due to the oxidation or reduction of the target analyte or an electroactive by-product of the target analyte. The electrical circuit 240 can be substantially similar to the electrical circuit 140 included in the electrochemical sensing system 100, and is therefore not described in further detail herein. The communications module 242 can be electrically coupled to the electrical circuit 240. The communications module 242 can be substantially similar to the communications module 142 included in the electrochemical sensing system 100, and is therefore not described in further detail herein.

The housing 250 includes a base portion 252 and a deformable portion 254. The housing 250 defines an internal volume 256 which is configured to house the components of the electrochemical sensing system 200. The deformable portion 254 can define a roof of the housing 250. While shown as having an arcuate shape, the deformable portion 254 can be substantially flat. The housing 250 can be formed from any suitable materials, or have any shape, as described with respect to housing 150 included in the electrochemical sensing system 100. The base portion 252 of the housing 250 can include an adhesive for removably associating the housing 250 to the body of a user, or any other suitable coupling mechanism as described with respect to housing 150.

The housing 250 can be configured to removably associate the working electrode 210 and the reference electrode 230 to a target, for example, the skin of the user. For example, in a first configuration shown in FIG. 2A, the working electrode 210 and the reference electrode 230 can be disposed substantially in the internal volume 256 defined by the housing 250 and the deformable portion 254 can be in an undeformed position. A user can exert a force in a direction shown by the arrow F₁ on the deformable portion 254. This can urge the electrochemical sensing system 200 to move into a second configuration shown in FIG. 2B. In the second configuration, the force F₁ can urge the deformable portion 254 to move into a deformed position. The deformable portion 254 can exert a force on the electrical circuit 240 urging the electrical circuit 240 to displace in a direction shown by the arrow A. The displacement of the electrical circuit 240 moves the working electrode 210 and the reference electrode 230 such that at least a portion of the working electrode 210 and the reference electrode 230 passes through the apertures 257 and 258, respectively and are disposed outside the internal volume 256. For example, the base portion 252 of the housing 250 can be disposed on the skin of a user so that in the second configuration, the working electrode 210 and the reference electrode 230 are inserted into the skin of the user and contact a bodily fluid, for example, blood. The working electrode 230 can then be biased at less than about 0.4 V to electrochemically sense the target analyte, as described herein. While the electrical circuit 240 is shown as being displaced in the second configuration, in some embodiments, the electrical circuit 240 can be fixedly disposed in the housing 250. In such embodiments, a transfer member, for example, a platform, a slab, or any other transfer member, can be coupled to the working electrode 210 and the reference electrode 230. The transfer member can be configured to displace and urge the working electrode 210 and the reference electrode 230 into the second configuration.

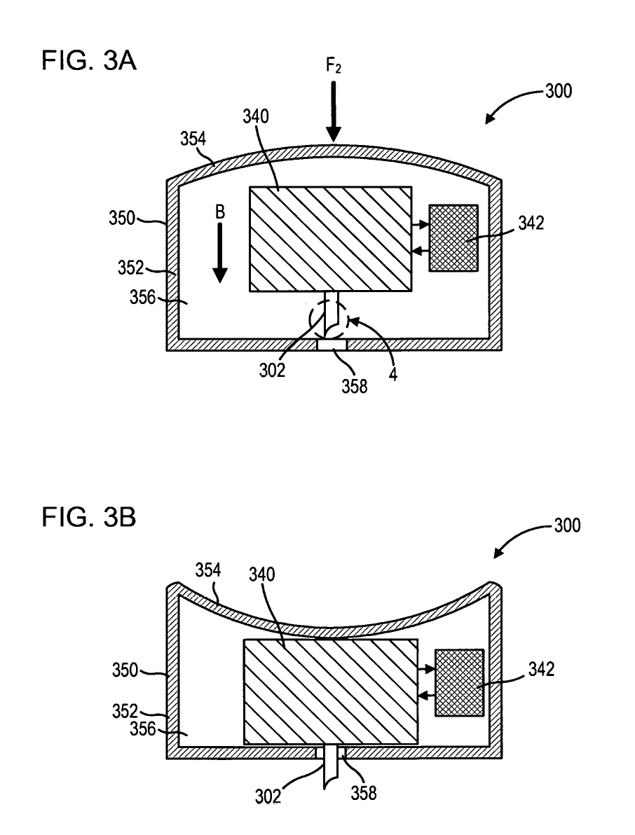

In some embodiments, an electrochemical sensing system can include a penetration member. Referring now to FIGS. 3A and 3B, an electrochemical sensing system 300 includes a penetration member 302, an electrical circuit 340 a communications module 342, and a housing 350. The electrochemical sensing system 300 can be configured to be associated with a target, for example, the skin of the user, such that the electrochemical sensing system 300 is wearable.

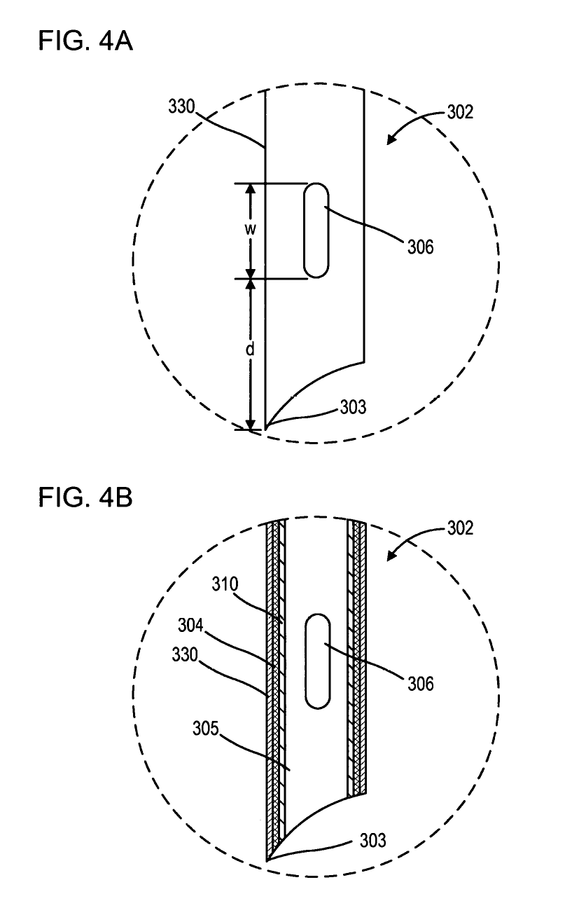

Referring also now to FIGS. 4A and 4B, the penetration member 302 is configured to penetrate the skin of a user and includes a working electrode 310 and a reference electrode 330. As shown in FIGS. 4A and 4B, the penetration member 302 includes a cylindrical member 302 that defines a lumen 305. A distal end of the penetration member 302 can forms a sharp tip 303, such that the penetration member 302 can resemble a needle (e.g., a 30 gage needle). This can enable the penetration member 302 to easily pierce into the skin of a user. The working electrode 310 can be disposed on an inner surface of the cylindrical member 304 that defines the lumen 305. The reference electrode 330 can be disposed on an outer surface of the cylindrical member 304. The cylindrical member 304 can be formed from a rigid and insulating material, for example, plastics, silicon oxide, silicon nitride, ceramics, or any other suitable insulating material. Alternatively, the cylindrical member 304 can be formed from a conductive material (e.g., metals) which is coated with an insulating layer to prevent electrical shorting of the working electrode 310 and the reference electrode 330. A set of apertures 306 are defined on a sidewall of the cylindrical member 302. The apertures 306 can, for example, allow air disposed within the lumen 305 to be expelled and allow a bodily fluid to flow in to the lumen 305. This can enable fluidic contact between the working electrode 310 and the reference electrode 330 such that electrochemical sensing of a target analyte included in the bodily fluid can be performed. The apertures 306 can be disposed at a distance d, for example, about 0.05 inches, from the distal end of the penetration member 302. The apertures 306 can be oblong and have a width w, for example, a width of about 0.025 inches. While shown as being oblong, the apertures 306 can have any shape, for example, circular, square, rectangular, elliptical, or any other suitable shape or size.

The working electrode 310 can be configured to oxidize a target analyte at a biasing voltage of less than about 0.4 V such that oxidation and reduction of one or more interfering species on the working electrode 310 is substantially reduced. The working electrode 310 can be formed from substantially the same materials and processes described with respect to the working electrode 110 included in the electrochemical sensing system 100. Furthermore, a biosensing molecule or any other functional layer can be disposed on the working electrode 310 as described with respect to the working electrode 110 included in the electrochemical sensing system 100. The reference electrode 330 is electronically coupled to the working electrode 310 via the electrical circuit 340. The reference electrode 330 is configured to provide a stable reference voltage for the working electrode 310 and does not get consumed by the oxidation or reduction reaction. The reference electrode 330 can be formed from the same materials and can include one or more functional layers as described with respect to the reference electrode 130 included in the electrochemical sensing system 100.

The electrical circuit 340 is configured to bias the working electrode 310 at a predetermined operating voltage, for example, a voltage of less than about 0.4 V and measure a redox current due to the oxidation or reduction of the target analyte or an electroactive by-product of the target analyte. The electrical circuit 340 can be substantially similar to the electrical circuit 140 included in the electrochemical sensing system 100, and is therefore not described in further detail herein. The communications module 342 can be electrically coupled to the electrical circuit 340. The communications module 342 can be substantially similar to the communications module 142 included in the electrochemical sensing system 100, and is therefore not described in further detail herein.

The housing 350 includes a base portion 352 and a deformable portion 354. The housing 350 defines an internal volume 356 which is configured to house the components of the electrochemical sensing system 300. The housing 350 can be substantially similar to the housing 250 described with respect to the electrochemical sensing system 200, and is therefore not described in further detail herein. In a first configuration, the penetration member 302 and thereby, the working electrode 310 and the reference electrode 330 are disposed substantially inside the internal volume 356. A user can exert a force in a direction shown by the arrow F₂ on the deformable portion 354 to deform the deformable portion 354 into a deformed position, thereby urging the electrochemical sensing system 300 into a second configuration shown in FIG. 3B. Said another way, the deformable portion 354 can exert a force on the electrical circuit 340 urging the electrical circuit 340 to displace in a direction shown by the arrow B. The displacement of the electrical circuit 340 urges the penetration member 302 to move from the first configuration into the second configuration such that at least a portion of the penetration member 302 passes through an aperture 358 defined in the base portion 352, and extends outside the internal volume 356. For example, the base portion 352 of the housing 350 can be disposed on the skin of a user such that at least a portion of the penetration member 302 can be inserted into the skin of the user in the second configuration. This enables the working electrode 310 and the reference electrode 330 disposed on the penetration member 302 to contact a bodily fluid. In this manner, an electrochemical measurement of a target analyte present in the bodily fluid can be performed.

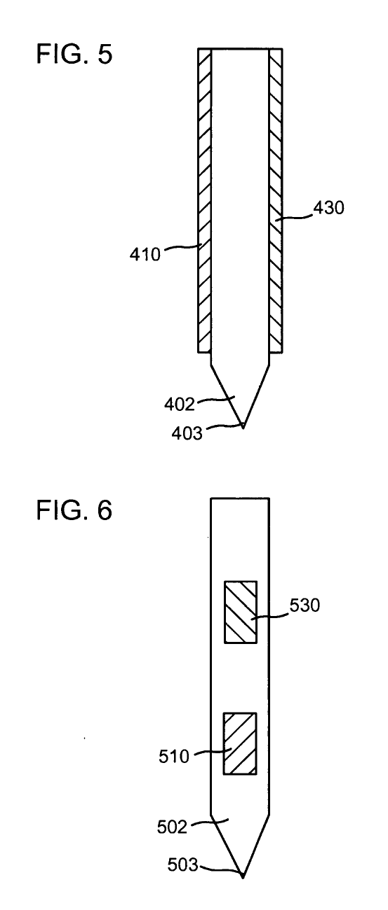

While shown as being a hollow member, in some embodiments the penetration member can be a solid. For example, FIG. 5 shows a penetration member 402 that includes a solid member (e.g., a cylinder). A distal end of the penetration member 402 forms a sharp tip 403 to facilitate insertion into the skin of a user. A working electrode 410 can be disposed on a first surface (e.g., a first side) of the penetration member 402 and the reference electrode 430 can be disposed on a second surface (e.g., a second side) of the penetration member 402. The penetration member 402 can be formed from an insulating material such as, for example, plastics, polyimide, silicones, silicon oxide, silicon nitride, ceramics, etc., or coated with an insulating material to prevent electrical shorting of the working electrode 410 and the reference electrode 430. The working electrode 410 and the reference electrode 430 can be substantially similar to the working electrode 310 and the reference electrode 330 described with respect to the penetration member 302, and are therefore not described in further detail herein.

In some embodiments, a working electrode and a reference electrode can both be disposed on the same surface of a penetration member. For example, FIG. 6 shows a penetration member 502 that includes a solid member (e.g., a cylinder) and defines a sharp tip 503. A working electrode 510 can be disposed on a first portion of a first surface and the reference electrode 530 can be disposed on a second portion of the first surface of the penetration member 502, such that the working electrode 510 and the reference electrode 530 are disposed along the same plane. The working electrode 510 and the reference electrode 530 can be substantially similar to the working electrode 310 and the reference electrode 330 described with respect to the penetration member 302, and are therefore not described in further detail herein.

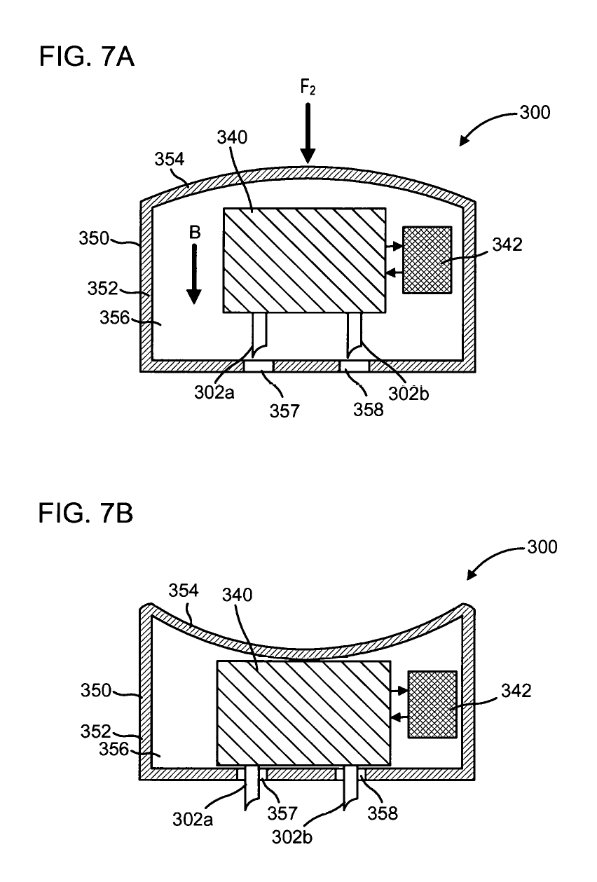

In some embodiments, an electrochemical sensing system 300 can include two penetration members. Referring now to FIGS. 7A and 7B, in some embodiments, the electrochemical sensing system 300 can include a first penetration member 302a and a second penetration member 302b which can be substantially similar to each other. Each of the first penetration member 302a and the second penetration member 302b can be substantially similar to any of the penetration member 302, 402 or 502 described herein, and are therefore not described in further detail herein. In some embodiments, the working electrode included in the first penetration member 302a can be configured to sense a first target analyte, and the working electrode included in the second penetration member 302b can be configured to sense a second target analyte different from the first target analyte. In some embodiments, the first working electrode and the second working electrode can include different sensing chemistries, for example, to reduce noise or eliminate calibration as described herein. In some embodiments, the first working electrode and the second working can have the same sensing chemistries and can be configured to sense the same target analyte. In such embodiments, the first working electrode included in the first penetration member can be biased at a first biasing potential (e.g., about +0.4 volts) and the second working electrode included in the second penetration member can be biased at a second biasing potential higher then the first biasing potential (e.g., about +0.7 volts). The first working electrode can measure a first signal that includes the redox current of the target analyte, and has a first magnitude. The second working electrode can measure a second signal that also includes the redox current of the target analyte. However because the second voltage is higher than the first voltage, the magnitude of the redox signal measured by the second electrode can be higher than the magnitude of the redox signal measured by the first electrode, such that the second signal has a second magnitude higher than the first magnitude. Since the first working electrode and the second working electrodes are substantially similar to each other, the signals measured by each electrode can also be substantially similar to each other and therefore, can be ratiometrically related to each other. In this manner, the need for external calibration can be eliminated, as described herein.

In a first configuration, the first penetration member 302a and the second penetration member 302b are disposed inside the internal volume 356. A user can exert a force in a direction shown by the arrow F₂ on the deformable portion 354 to deform the deformable portion 354 into a deformed position, thereby urging the electrochemical sensing system 300 into a second configuration shown in FIG. 7B. Said another way, the deformable portion 354 can exert a force on the electrical circuit 340 urging the electrical circuit 340 to displace in a direction shown by the arrow B. The displacement of the electrical circuit 340 urges the first penetration member 302a and the second penetration member 302b to move from the first configuration into the second configuration such that at least a portion of the first penetration member 302a and the second penetration member 302b pass through a first aperture 357 and a second aperture 358, respectively defined in the base portion 352, and extend outside the internal volume 356. For example, the base portion 352 of the housing 350 can be disposed on the skin of a user such that at least a portion of the first penetration member 302a and the second penetration member 302b can be inserted into the skin of the user in the second configuration. In this manner, an electrochemical measurement of a target analyte (or a plurality of target analytes) present in the bodily fluid can be performed.

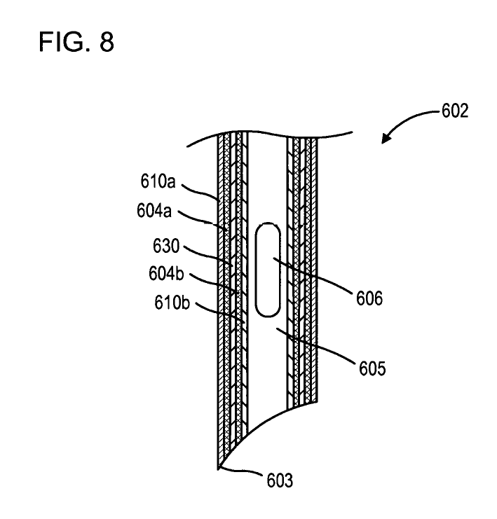

While shown as having, a first penetration member 302a and a second penetration member 302b, in some embodiments, the electrochemical sensing system 300 can include a single penetration member that includes a first working electrode and a second working electrode. For example, FIG. 8 shows a side-cross section of a penetration member 602 that can be included in the electrochemical sensing system 300 or any other electrochemical sensing system described herein. The penetration member 602 can be a substantially cylindrical member that defines a lumen 605. A distal end of the penetration member 602 can form a sharp tip 603, such that the penetration member 302 can resemble a needle (e.g., a 30 gage needle). This can enable the penetration member 602 to easily pierce into the skin of a user. A first working electrode 610a can be disposed on an outer surface of the penetration member 602 and a second working electrode 610b can be disposed on an inner surface of the penetration member 602. A reference electrode 630 can be disposed in between the first working electrode 610a and the second working electrode 610b. The reference electrode 630 is electrically isolated from the first working electrode 610a by a first insulating layer 604a. Similarly, the reference electrode 630 is isolated from the second working electrode 610b by a second insulating layer 604b. The penetration member 602 can be formed using any suitable process, for example, co-extrusion, metal deposition, electroplating, any other suitable process or combination thereof.

One or more apertures 606 are defined on a sidewall of the penetration member 602. The apertures 606 can, for example, allow air disposed within the lumen 605 to be expelled and allow a bodily fluid to flow in to the lumen 605. This can enable fluidic contact between the first working electrode 610a, the second working electrode 610b, and the reference electrode 630 such that electrochemical sensing of one or more target analytes included in the bodily fluid can be performed. Each of the first working electrode 610a and the second working electrode 610b can be paired with the reference electrode 630. In some embodiments, the first working electrode 610a and the second working electrode 610 can include different sensing chemistries, for example, to sense different target analytes or to reduce noise, as described herein. In some embodiments, the first working electrode 610a and the second working electrode 610b can be configured to sense the same analyte and can be polarized at different biasing voltages, such that no external calibration is required, as described herein.

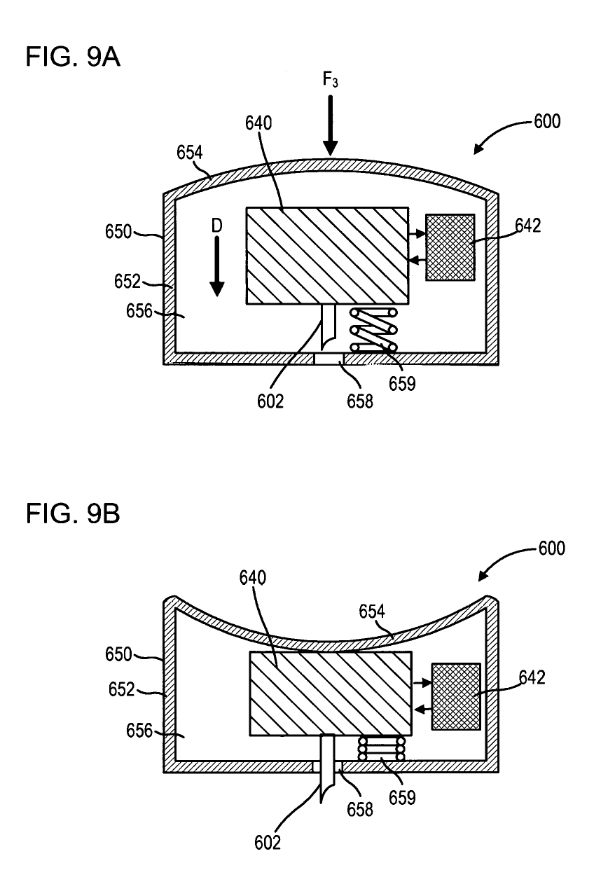

In some embodiments, an electrochemical sensing system can include a retraction mechanism. Referring now to FIGS. 9A and 9B, an electrochemical sensing system 600 includes the penetration member 602, an electrical circuit 640 a communications module 642, a housing 650, and a retraction mechanism 659. The electrochemical sensing system 600 can be configured to be associated with a target, for example, the skin of the user, such that the electrochemical sensing system 600 is wearable.

The penetration member 602 is configured to penetrate the skin of a user and includes a first working electrode, a second working electrode, and a reference electrode, as described in detail herein. While shown as including the penetration member 602, in some embodiments, the electrochemical sensing system can include any other penetration member, for example the penetration member 302, 402, 502 or any other penetration member described herein.

The working electrode can be configured to oxidize a target analyte at a biasing voltage of less than about 0.4 V such that oxidation and reduction of one or more interfering species on the working electrode is substantially reduced. The working electrode can be substantially similar to the working electrode 110, 210, 310, or any other working electrode described herein, and is therefore not described in further detail herein. The reference electrode is electronically coupled to the working electrode via the electrical circuit 640. The reference electrode can be substantially similar to the reference electrode 130, 230, 330, or any other working electrode described herein, and is therefore not described in further detail herein.

The electrical circuit 640 is configured to bias the working electrode at a predetermined operating voltage, for example, a voltage of less than about 0.4 V and measure a redox current due to the oxidation or reduction of the target analyte or an electroactive by-product of the target analyte. The electrical circuit 640 can be substantially similar to the electrical circuit 140 included in the electrochemical sensing system 100, and is therefore not described in further detail herein. The communications module 642 can be electrically coupled to the electrical circuit 640. The communications module 642 can be substantially similar to the communications module 142 included in the electrochemical sensing system 100, and is therefore not described in further detail herein.

The housing 650 includes a base portion 652 and a deformable portion 654 defining an internal volume 656 therebetween which is configured to house the components of the electrochemical sensing system 600. The housing 650 can be substantially similar to the housing 250 described with respect to the electrochemical sensing system 200, and is therefore not described in further detail herein. A retraction mechanism 659 can be disposed in the housing 650 for example, disposed on an inner surface of the base 652 of the housing 650. The retraction mechanism 659 can include, for example, a biasing member such as, for example, a spring (e.g., compression, extension, torsion, spring washers, Belleville, tapered, any other type of spring) any other suitable biasing member or a combination thereof.

The retraction mechanism 659 can be configured to retract the penetration member 602 into internal volume 656 after at least a portion of the penetration member 602 has been disposed outside the internal volume 656. For example, in a first configuration shown in FIG. 9A, the penetration member 602 can be disposed substantially in the internal volume 656 defined by the housing 650 and the deformable portion 654 can be in an undeformed position. A user can exert a force in a direction shown by the arrow F₃ on the deformable portion 654. This can urge the electrochemical sensing system 600 to move into a second configuration shown in FIG. 9B. In the second configuration, the force F₃ can urge the deformable portion 654 to move into a deformed position. The deformable portion 654 can exert a force on the electrical circuit 240 urging the electrical circuit 640 to displace in a direction shown by the arrow D. The displacement of the electrical circuit 640 moves the penetration member 602 such that at least a portion of the penetration member 602 passes through the aperture 658, respectively and is disposed outside the internal volume 656. For example, the base portion 652 of the housing 650 can be disposed on the skin of a user so that in the second configuration, the working electrode and the reference electrode included in the penetration member 602 are inserted into the skin of the user and contact a bodily fluid, for example, interstitial fluid.

The displacement of the electrical circuit 640 can also bias the retraction mechanism 659 in the second configuration, as shown in FIG. 9B. Once the electrochemical measurement is performed, the user can remove the force F₃. The retraction mechanism 659 can then urge the electrical circuit 640 to displace in a direction opposite to the direction shown by the arrow D. This can urge the deformable portion 654 to move into the undeformed position and the penetration member 602 to retract into the internal volume 656, until the penetration member 602 is disposed substantially inside the internal volume 656. In this manner, the penetration member 602 can return to the first configuration. In some embodiments, the retraction mechanism 659 can be configured to retract after a period of time. For example, once the user removes the force F₃, there can be a time delay before the retraction mechanism 659 activates and urges the electrical circuit 640 to move the deformable portion 654 into the undeformed position and retract the penetration member 602 into the internal volume 656. In some embodiments, the retraction mechanism 659 can be configured to retract gradually over a period of time. For example, after the user removes the force F₃, the retraction mechanism 659 can retract gradually over a period of time to move the deformable portion 654 into the undeformed position and retract the penetration member 602 into the internal volume 656. In such embodiments, the period of time can be sufficient for the working electrode and the reference electrode to perform an electrochemical measurement.

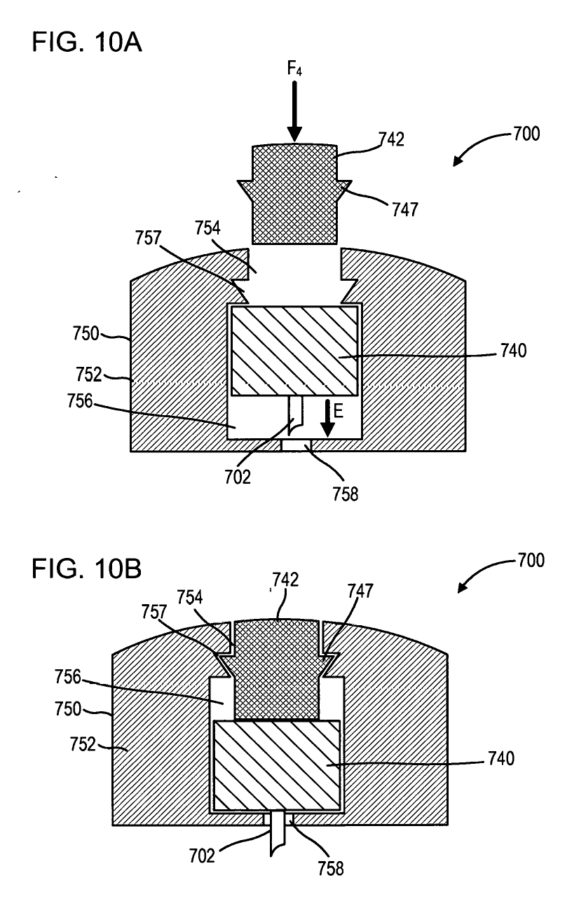

In some embodiments, an electrochemical sensing system can include a removable communications module. Referring now to FIGS. 10A and 10B, an electrochemical sensing system 700 includes a penetration member 702, an electrical circuit 740 a communications module 742, and a housing 750. The electrochemical sensing system 700 can be configured to be associated with a target, for example, the skin of the user, such that the electrochemical sensing system 700 is wearable.

The penetration member 702 is configured to penetrate the skin of a user and includes a working electrode and a reference electrode. The penetration member 702 can be substantially similar to the penetration member 302, 402, 502 or any other penetration member described herein, and is therefore not described in further detail herein.

The working electrode can be configured to oxidize a target analyte at a biasing voltage of less than about 0.4 V such that oxidation and reduction of one or more interfering species on the working electrode is substantially reduced. The working electrode can be substantially similar to the working electrode 110, 210, 310, or any other working electrode described herein, and is therefore not described in further detail herein. The reference electrode is electronically coupled to the working electrode via the electrical circuit 740. The reference electrode can be substantially similar to the reference electrode 130, 230, 330, or any other working electrode described herein, and is therefore not described in further detail herein.

The electrical circuit 740 is configured to bias the working electrode at a predetermined operating voltage, for example, a voltage of less than about 0.4 V and measure a redox current due to the oxidation or reduction of the target analyte or an electroactive by-product of the target analyte. The electrical circuit 740 can be substantially similar to the electrical circuit 140 included in the electrochemical sensing system 100, and is therefore not described in further detail herein.

The communications module 742 can be removably disposed in the housing and coupled to the electrical circuit 740. The communications module 642 can be substantially similar to the communications module 142 included in the electrochemical sensing system 100. The communications module 742 can include coupling members 747 such as, for example, notches, grooves, indents, a locking member, an other coupling members or combination thereof, which are configured to engage coupling features 757, for example, notches, grooves, indents, or combination thereof, included in a lumen 754 defined by the housing 750, as described herein.

The housing 750 includes a rigid base portion 752 that defines an internal volume 756. The penetration member 702 and the electrical circuit 740 can be slidably disposed in the internal volume 756. The housing 750 can be formed from any materials and can have any shape and size, as described with respect to the housing 150 included in the electrochemical sensing system 100, described herein. The housing 750 defines a recess 754, which is configured to receive the communications module 742. Coupling features 757 are disposed on a side wall of the recess 754, and are configured to engage the coupling members 747 included in the communications module 742, and removably couple the communications module 742 to the housing 750. In a first configuration, the communications module 742 can be disposed substantially outside the recess 754 and the penetration member 702 can be disposed substantially inside the internal volume 756. A user can insert the communications module 742 into the recess 754 in the direction shown by the arrow F₄. The communications module 742 can engage the electrical circuit 740 and establish electrical communication. The force of insertion (indicated by arrow F₄) can urge the electrical circuit 740 and the penetration member 702 coupled thereto, to displace in a direction shown by the arrow E such that in a second configuration, at least a portion of the penetration member 702 is disposed outside the internal volume 756. In the second configuration, the engaging members 747 included in the communications module 742 can engage the engaging features 757 includes in the recess 754 such that the communications module 742 is coupled to the recess 754 and remains disposed in the recess 754 after the force F₄ is removed. In some embodiments, the communications module 742 can be fixedly coupled to the housing 750 in the second configuration. In some embodiments, the communications module 742 can be releasably coupled to the housing 750 (e.g., to be reused with other sensing systems). While shown as having coupling features 747, in some embodiments, the communications module 742 can be coupled the housing 750 using a friction fit mechanism. In some embodiments, a retraction mechanism, for example, the retraction mechanism 659 can be included in the electrochemical sensing system 700. In such embodiments, the retraction mechanism can be configured to urge the penetration member 702 into the first configuration when the communications module 742 is uncoupled and removed from the recess 754.

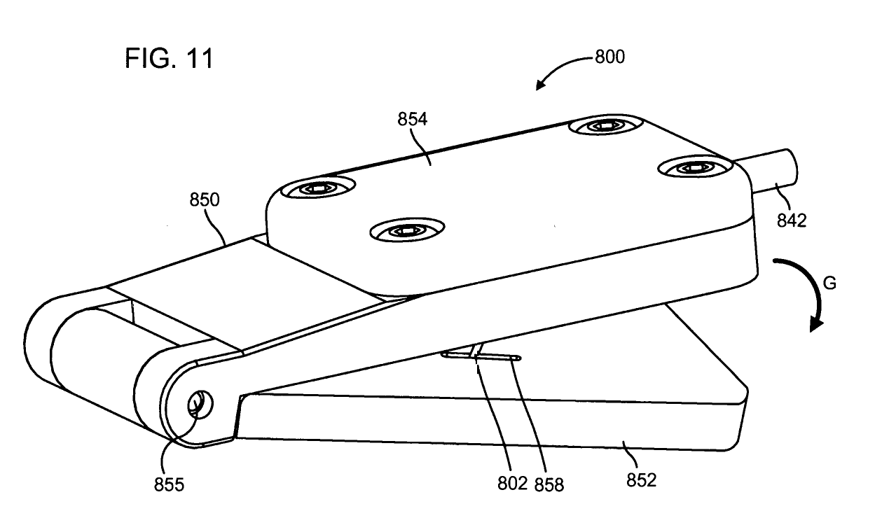

In some embodiments, an electrochemical sensing system can include a housing which is configured to removably associate a working electrode and a reference electrode with the body of a user. Referring now to FIGS. 11-13, an electrochemical sensing system 800 includes a penetration member 802 and a housing 850. The housing 850 includes a base portion 852 and a movable portion 854. The electrochemical sensing system 800 can be configured to be associated with a target, for example, the skin of the user, such that the electrochemical sensing system 800 is wearable.

The penetration member 802 is configured to penetrate the skin of a user and includes a working electrode and a reference electrode. The penetration member 802 can be substantially similar to the penetration member 302, 402, 502 or any other penetration member described herein, and is therefore not described in further detail herein.

An electrical circuit can be disposed in the base portion 852 or the movable portion 854 of the housing. The electrical circuit can be configured to bias the working electrode at a predetermined operating voltage, for example, a voltage of less than about 0.4 V and measure a redox current due to the oxidation or reduction of the target analyte or an electroactive by-product of the target analyte. The electrical circuit can be substantially similar to the electrical circuit 140, 240, 340 or any other electrical circuit described herein, and is therefore not described in further detail herein.

The communications module 842 can be disposed in the movable portion 854 of the housing. The communications module 842 can be substantially similar to the communications module 142 included in the electrochemical sensing system 100, and is therefore not described in further detail herein. While shown as having a portion of the communications module 842 disposed outside the movable portion 854, in some embodiments, the communications module 842 can be disposed substantially inside the movable portion 854.

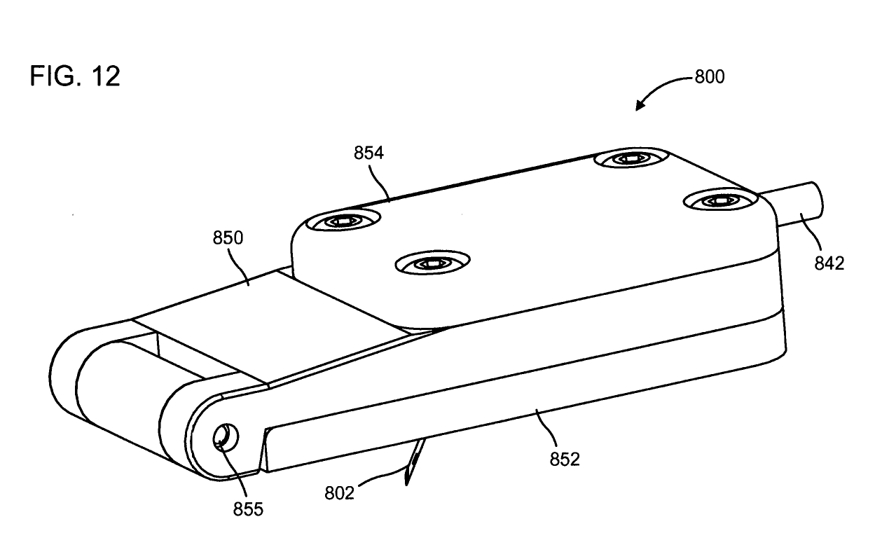

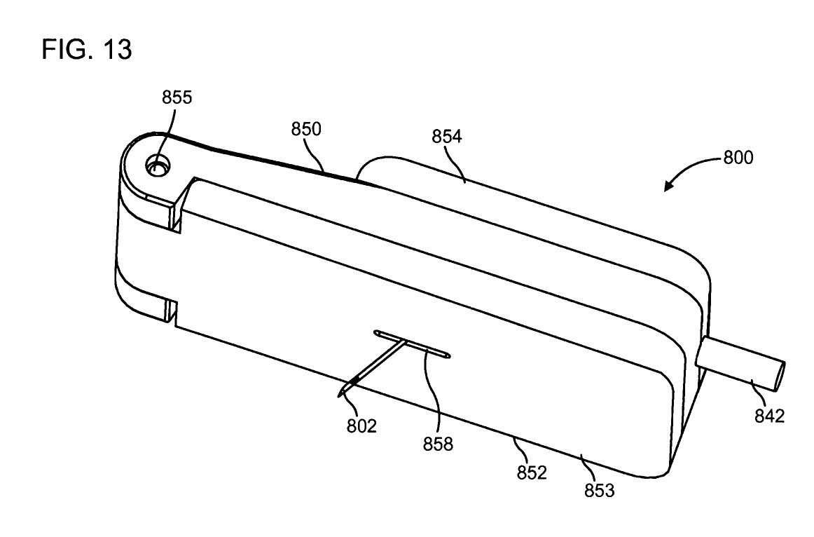

A bottom surface 853 of the base portion 852 (FIG. 13) can be disposed on the skin of the user. The bottom surface 852 can include an adhesive or any other suitable coupling mechanism for removably coupling the housing 850 to the skin of the user, as described with respect to the housing 150 included in the electrochemical sensing system 100. The movable portion 854 can be pivotally mounted to the base portion 852 at the hinges 855. The movable portion 854 can be configured to be movable between an unassembled position and an assembled position by moving the movable portion 854 with reference to the base portion 852. For example, in a first configuration shown in FIG. 11, the movable portion 854 can be in an unassembled position. In this position, the penetration member 802 is disposed within the housing, for example partially disposed inside an aperture 858 included in the base portion 852 such that no portion of the penetration member 802 is disposed outside the housing 850. A user can move the movable portion 854 by rotating the movable portion 854 about the hinges 855 in a direction shown by the arrow G. This can move the movable portion 854 into an assembled position (FIG. 12 and FIG. 13), urging the penetration member 802 into a second configuration. In the second configuration, at least a portion of the penetration member 802 can emerge through the aperture 858 defined in the base portion 852, and be disposed outside the housing 850. In this manner, the penetration member can be inserted into the skin of the user. To retract the penetration member 802, the movable portion 854 can be moved into the unassembled position. This can, for example, allow replacement of one or more components of the electrochemical sensing system 800, for example, the penetration member 802.

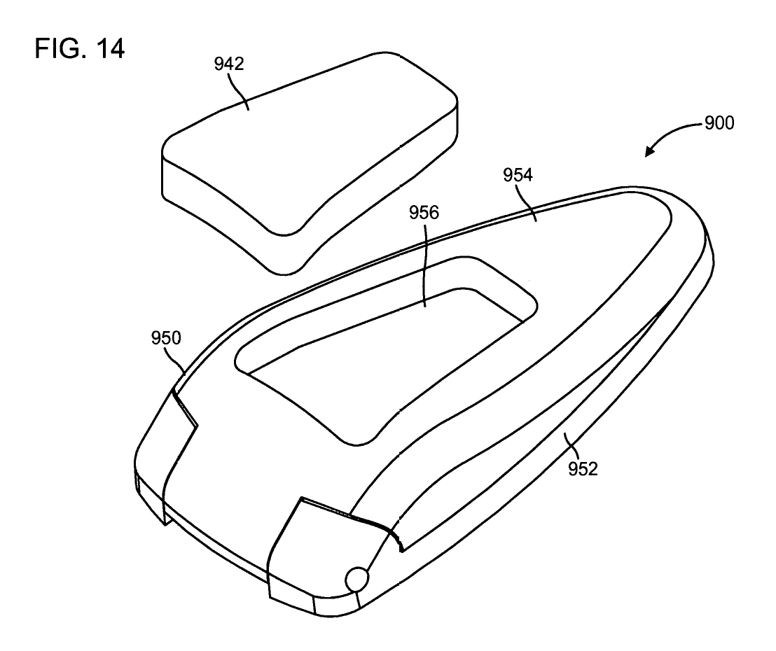

In some embodiments, an electrochemical sensing system can include a removably coupled communications module. Referring now to FIGS. 14-16, an electrochemical sensing system 900 includes a penetration member 902, a communications module 942, and a housing 950. The housing 950 includes a rigid base portion 952 and a rigid cover portion 954. The electrochemical sensing system 900 can be configured to be associated with a target, for example, the skin of the user, such that the electrochemical sensing system 900 is wearable.

The penetration member 902 is configured to penetrate the skin of a user and includes a working electrode and a reference electrode. The penetration member 902 can be substantially similar to the penetration member 302, 402, 502 or any other penetration member described herein, and is therefore not described in further detail herein.

An electrical circuit can be disposed in the base portion 952 of the housing 950. The electrical circuit can be configured to bias the working electrode at a predetermined operating voltage, for example, a voltage of less than about 0.4 V and measure a redox current due to the oxidation or reduction of the target analyte or an electroactive by-product of the target analyte. The electrical circuit can be substantially similar to the electrical circuit 140, 240, 340 or any other electrical circuit described herein, and is therefore not described in further detail herein.

The communications module 942 can be removably disposed in the cover portion 954, as described herein. The communications module 942 can be substantially similar to the communications module 142, 242, 742, or any other communications module described herein.

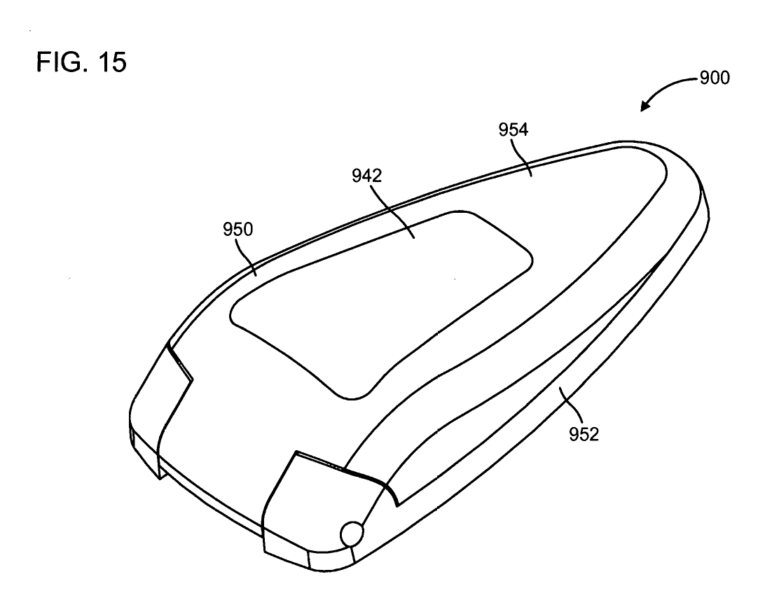

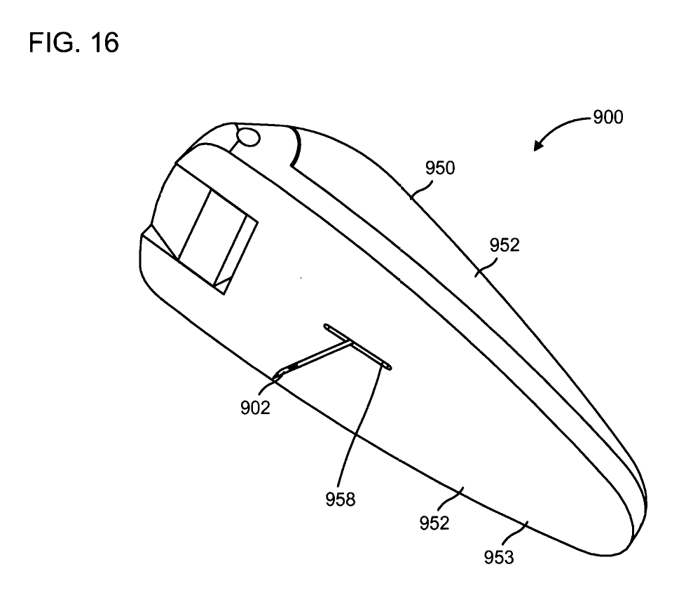

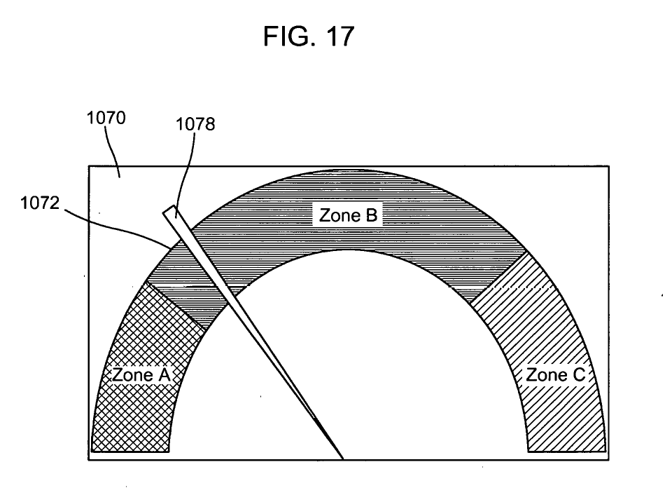

The base portion 952 and the cover portion 954 included in the housing 950 can define an internal volume therebetween within which one or more components of the electrochemical sensing system 900 can be disposed. A bottom surface 953 of the base portion 952 (FIG. 16) can be disposed on the skin of the user. The bottom surface 953 can include an adhesive or any other suitable coupling mechanism for removably coupling the housing 950 to the skin of the user, as described with respect to the housing 150 included in the electrochemical sensing system 100. The cover portion 954 can be fixedly coupled to the base portion 952. The cover portion 954 can include an arcuate surface such that the cover portion 954 defines a dome shape and the housing 950 resembles a tear drop shape. The cover portion 954 defines a cavity 956 configured to matingly receive the communications module 942. Disposing the communications module 942 in the cavity 956 can urge at least a portion of the penetration member 902 to emerge from the aperture 953 defined in the base portion 952 (FIG. 17) and, for example, be inserted into the skin of the user. For example, in a first configuration shown in FIG. 14, the communications module 942 is disposed outside the cavity 956 and the penetration member 902 is disposed substantially inside the housing 950. A user can insert the communications module 942 into the cavity 956. The communications module 942 and the cavity 956 can include coupling features such as, for example, notches, grooves, indents, snap-fit mechanism, or any other coupling mechanism to removably coupled the communications module 942 to the cover portion 952. The communications module 942 can include an arcuate surface, such that top edges of the communications module 942 can be flush with the top edges of the cavity 956 and the exposed surface of the communications module 942 also defines a dome shape. The communications module 942 is configured, such that insertion of the communications module 942 into the cavity 956 (FIG. 15), moves the penetration member 902 into a second configuration (FIG. 16), such that at least a portion of the penetration member 902 passes through an aperture 958 and is disposed outside the housing 950. In this manner, the penetration member 902 can be inserted into the skin of the user. In some embodiments, a retraction mechanism, for example, a biasing member (e.g., as spring such as, for example, a compression, extension, torsion, spring washers, Belleville, tapered, any other type of spring) or any other retraction mechanism can be disposed inside the housing 950. For example, the retraction mechanism can be configured to urge the penetration member into the first configuration, when the communications module 942 is removed from the cavity 956. This can, for example, allow replacement of one or more components of the electrochemical sensing system 900, for example, the penetration member 902.

In some embodiments, a communications module included in an electrochemical sensing system, for example, the electrochemical sensing system 100, 800, 900 or any other electrochemical sensing system described herein can include a dial gage display. FIG. 17 shows a display 1070 that can be included in any of the communication modules described herein. The display 1070 includes a static dial gage 1072 and a movable indicator 1078, for example, a digital needle. The dial gage 1072 includes three color coded zones. Zone A is red, zone B is green, zone C is color coded yellow. Each zone can correspond to a specific concentration range, band, or range of the target analyte. In some embodiments, the color coded zones can be configured to indicate a physiological state of a user. For example, in some embodiments, the target analyte can be glucose and the color coded zones can indicate regions of hypoglycemia, hyperglycemia, or euglycemia to the user. Furthermore, the displacement speed of the indicator 1078 can be used to indicate the rate of change of the concentration of the target analyte (e.g., glucose). For example, fast or slow ghosting (i.e., movement) of the indicator 1078 towards a first side of the dial gage 1072 (e.g., zone C) can indicate that the concentration of the target analyte is rising fast or slow, respectively. Similarly, fast or slow ghosting (i.e., movement) of the indicator 1078 towards a second side of the dial gage 1072 (e.g., zone A) can indicate that the concentration of the target analyte is falling fast or slow, respectively. No movement of the indicator 1078 (e.g., static in zone B) can correspond to a stable concentration of the target analyte.

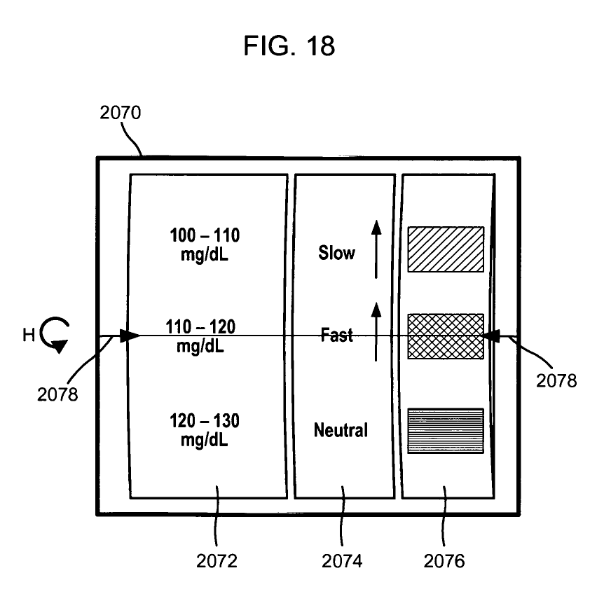

In some embodiments, a communications module can include a wheel type display (e.g., a digital wheel display). Referring now to FIG. 18, a display 2070 includes a first wheel 2072, a second wheel 2074, a third wheel 2076, and a static indicator 2078. Each of the wheels can be movable and can be configured to rotate in a clockwise or counter clockwise direction, as shown by the arrow H. The information on each of the wheel that aligns with the indicator 2078 provides the user information on the concentration of the target analyte, rate of change of the target analyte, and a physiological state of the user. For example, the first wheel 2072 can be configured to display information on the concentration of the target analyte, concentration ranges, bands or values. The second wheel 2074 can be configured to display information on the rate of change of the concentration of the target analyte. For example, the second wheel 2074 can display the word include, “Slow”, and an upward or downward pointing arrow to indicate slow rise or fall of the target analyte concentration, respectively. Similarly, the second wheel 2074 can display the word, “Fast”, and an upward or downward pointing arrow to indicate fast rise or fall of the target analyte concentration, respectively. The word “neutral” can be used to indicate no change in the target analyte concentration. The third wheel 2076 can be configured to display information on a physiological status of the user and can include color coded zones, for example, a red, yellow, and green, or any other color coded zones. Each zone can correspond to a physiological status of the user. For example, the target analyte can be glucose and the color coded zones can be configured to indicate hypoglycemia, hyperglycemia, and euglycemia. While the indicator 2078 is shown as including two arrows connected by a straight line, in some embodiments, the indicator 2078 can be configured to highlight a background on each of the wheels to indicate the target analyte data.

The wearable electrochemical sensing systems described herein, for example, the electrochemical sensing system 100, 200 800, 900, or any other electrochemical sensing systems described herein, can be used to provide a user real time information on the concentration of a target analyte within a bodily fluid, for example, blood. For example, in some embodiments, the target analyte can be glucose. Any of the wearable electrochemical sensing systems described herein can be used to monitor the glucose concentration in a bodily fluid, for example, the blood of the user. The electrochemical sensing system (e.g., any of the electrochemical sensing systems described herein) can inform the user on the real time level of blood glucose. The user, for example, a diabetic can use this information to control his blood sugar and make better food choices. In some embodiments, real time glucose concentration data measured by any of the electrochemical sensing systems described herein can, for example, be used to help a user in determining exercise intensity, aid in recovery from low glucose, low or high glucose prevention, monitoring daily glucose levels to better manage weight loss, or any other health management program. The following are various examples of using real time glucose monitoring data measured by any of the wearable electrochemical sensing systems described herein, to display a user desired information, or information which the user can employ to better manage overall health and well being.

### Exercise Intensity

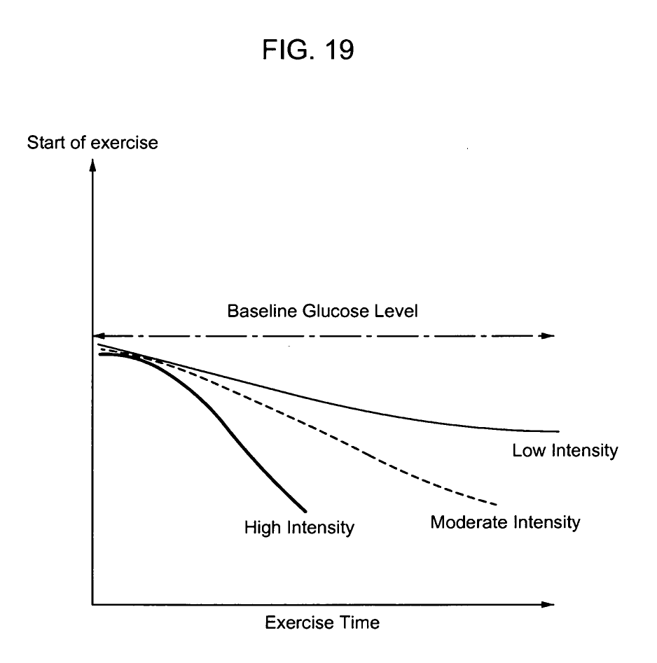

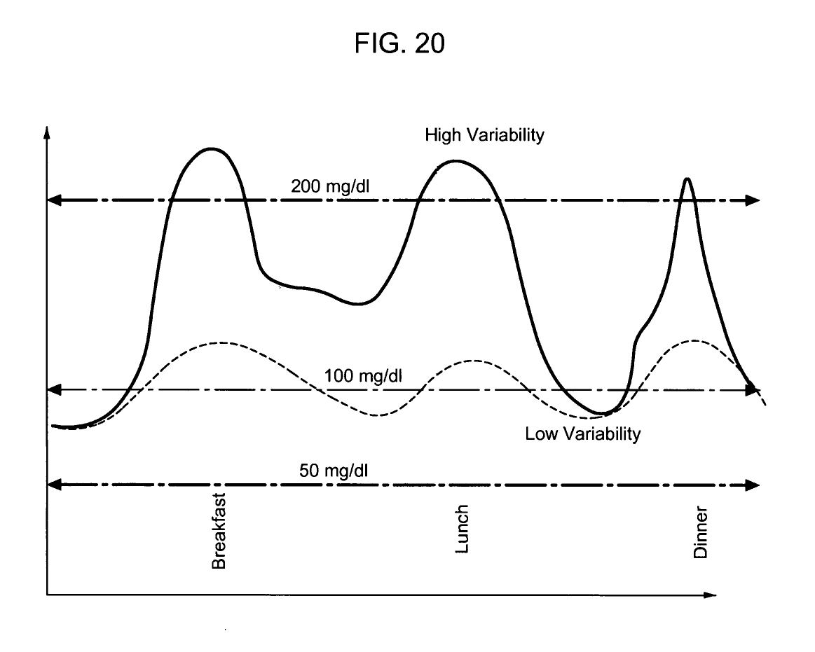

In some embodiments, the wearable electrochemical sensing systems described herein which are configured to measure glucose can be used to determine an intensity of exercise of a user (also referred to as the “GLUCOSE RATE INDEX™). For example, FIG. 19 shows a plot depicting the intensity of exercise performed by a user based on the glucose level measured by the wearable electrochemical sensing system. In this scenario, the user's blood glucose level can be used as a direct indicator of the user's metabolic activity. The rate of change of the user's blood glucose concentration is used to determine the exercise intensity and metabolic demand. As shown in FIG. 19, a faster and deeper drop in blood glucose level predicts a higher intensity of the exercise. This can be used to prevent a sudden drop in metabolism due to a drop in blood glucose level which can lead to feeling of lethargy and sudden drop in exercise intensity.

### Optimize Recovery—Low Glucose Prevention

The real time glucose data measured by the wearable electrochemical sensing systems described herein can be used to aid a user in recovery. Athletes often experience low glucose levels during exercise. Dietary choices can also impact glucose levels post exercise (e.g., drinking large quantities of fluid replacement drinks post exercise). These low glucose levels can impact glucose uptake into muscles for hours to days and can also impact the replenishment of muscle glycogen storage. The glucose level data can help the user in selecting a post exercise meal, snack, or fluid replacement regimen in order to optimize the body's ability to restore muscle glycogen.

### Health Wellness Indicator

Glucose variability, i.e. highs and lows in glucose over a period of time (also referred to as the GLUCOSE VARIATION INDEX™) is a measure of the wellness of the user. Excessive glucose in the blood and high glucose excursions have been linked to a number of a health issues. The higher the glucose variation the worse the index and the more likely that long term complications will result from poor diet and exercise. The lower the variability the better the diet and exercise programs and the better the likelihood of good future health. This information can benefit pre-diabetics allowing them to understand and keep track of the level of glucose variations in their body. FIG. 18 shows a sample GLUCOSE VARIATION INDEX™ of a user over a period of 24 hours.

### Weight Loss

Real time glucose monitoring performed by the wearable electrochemical sensing systems described herein can also be used to manage a weight loss regimen. For example, the glucose monitoring data can be used to determine a TOTAL DAILY GLUCOSE INDEX™. This index can either be a glucose deficit (weight loss) or glucose surplus (weight gain) and can be determined from the area under the curve compared to the fixed glucose. A measurement below a certain threshold (glucose deficit) can indicate that the user is losing weight, and a measurement above the threshold (glucose surplus) can indicate that the user is gaining weight. This data can be used in combination with glucose variability metrics to give the user a better understanding of their individual metabolic profile.

In some embodiments, the index can be calculated for each individual meal (also referred to as “the GLUCOSE MEAL INDEX™”) to create a metric on any meal in real time. This can provide the user feedback on the user's diet and meal selection and how the user's body responds at any given time since several other parameters other than glucose can also influence the body's response to food. A user can eat the same meal at two different times and have two different glucose profiles due to various physiological factors. Additionally, low glucose prevention can help limit cravings in a user who is trying to practice a dieting regimen.

Combining Glucose Data with Other Physiological Data

In some embodiments, the glucose monitoring data of a user can be combined with other physiological monitoring data, for example, activity monitoring data (e.g., heart rate, steps taken, running speed, pulse rate, body temperature, etc.) to create a complete health profile of the user. For example, heart rate data can be combined with the glucose intensity index to give a better picture of the athletic exertion of the user and can provide even an average athlete a new target during exercise. In some embodiments, activity monitoring sensors, for example, pulse rate sensors, oxygen sensors, temperature sensor, any other activity sensors or combination thereof, can be included in the electrochemical sensing systems described herein.

The glucose monitoring data can be used to provide a better picture of calories burned by a user during exercise. Heart rate by itself is a poor indicator of calories burned during low to moderate intensity exercise and can often result in a massive over prediction of the calories consumed. The combination of the glucose (either using the GLUCOSE RATE INDEX™ or TOTAL DAILY GLUCOSE INDEX™) and heart rate could be used to enhance the correlation of calories burned to moderate exercise intensity. This information can be also be used with activity trackers.

Furthermore, the glucose monitoring data can be used for prediction of “afterburn” which can be experienced by a user post a vigorous short duration exercise. The afterburn has been correlated to higher oxygen consumption. Thus the glucose monitoring data and particularly the TOTAL DAILY GLUCOSE INDEX™ can be used as a direct measure of this event and can show the impact of high intensity short duration workouts on fat burning.

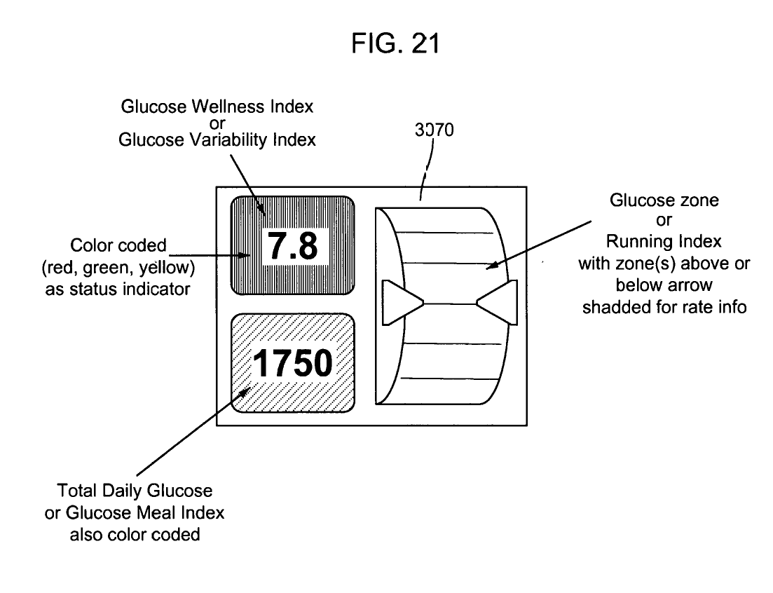

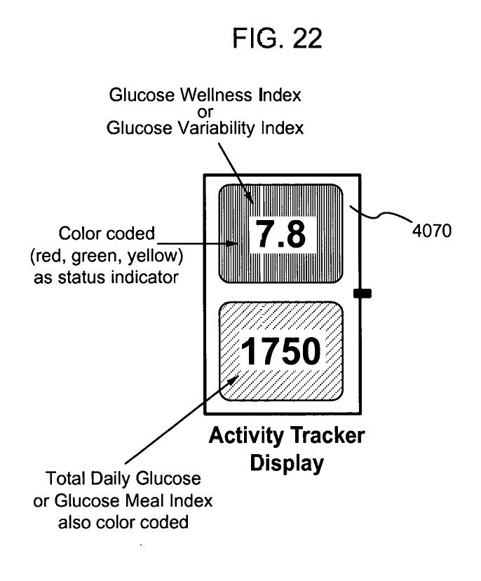

In some embodiments, the wearable electrochemical sensing system 100, 200, 300, 400, 500, 600, 700, or any other electrochemical sensing system described herein, can include a display to communicate information on the indices described herein to the user. For example, FIG. 21 shows a display 3070 that can be included in any of the electrochemical sensing systems described herein. The display 3070 includes a wheel type gage configured to communicate information on a glucose zone (or running index) to the user. The zones above or below the indicator can be shaded to communicate rate information to the user. The display 3070 also includes a first digital display and a second digital display for communicating information on the GLUCOSE VARIATION INDEX™ and TOTAL DAILY GLUCOSE INDEX™ (or GLUCOSE MEAL INDEX™). Furthermore, each of the first and second digital display can be color coded (e.g., red, green, blue, yellow) to indicate a status of the user. In some embodiments, a wearable electrochemical sensing system which includes the display 3070 can be configured to resemble a wrist watch, for example, to be worn on the wrist of the user. In some embodiments, an electrochemical sensing system, for example, any of the electrochemical sensing systems described herein can be configured as an activity tracker. In such embodiments, the electrochemical sensing system can include a display configured to communicate information only on the GLUCOSE VARIATION INDEX™ and the TOTAL DAILY GLUCOSE INDEX™ (or GLUCOSE MEAL INDEX™). FIG. 22 shows a display 4070 that can be included in a wearable electrochemical sensing system configured to track an activity of a user. The display 4070 also includes a first digital display and a second digital display for communicating information on the GLUCOSE VARIATION INDEX™ and TOTAL DAILY GLUCOSE INDEX™ (or GLUCOSE MEAL INDEX™). Furthermore, each of the first and second digital display can be color coded (e.g., red, green, blue, yellow) to indicate a status of the user.

While various embodiments of the system, methods and devices have been described above, it should be understood that they have been presented by way of example only, and not limitation. Where methods and steps described above indicate certain events occurring in certain order, those of ordinary skill in the art having the benefit of this disclosure would recognize that the ordering of certain steps may be modified and such modification are in accordance with the variations of the invention. For example, the non-aqueous electrolyte can also include a gel polymer electrolyte. Additionally, certain of the steps may be performed concurrently in a parallel process when possible, as well as performed sequentially as described above. The embodiments have been particularly shown and described, but it will be understood that various changes in form and details may be made.

1. An electrochemical sensing system, comprising:
a housing configured to be removably associated with a user;
a working electrode at least partially disposed in the housing, at least a portion of the working electrode including rhodium metal;
a reference electrode at least partially disposed in the housing;
an electrical circuit disposed in the housing and configured to be electronically coupled to the working electrode and the reference electrode, the electrical circuit operable to: (a) bias the working electrode at a voltage of less than about 0.4 V such that a target analyte decomposes, and (b) measure a current corresponding to the concentration of the target analyte; and
a communication module coupled to the electrical circuit and configured to at least one of display a concentration of the target analyte, and communicate data between the electrical circuit and an external device, the data including the concentration of the target analyte,
the working electrode and the reference electrode movable between a first configuration in which the working electrode and the reference electrode are disposed substantially inside the housing, and a second configuration in which at least a portion of the working electrode and a portion of the reference electrode are disposed outside the housing.

2. The electrochemical system of claim 1, further comprising:
an insertion mechanism configured to move the working electrode and the reference electrode between the first configuration and the second configuration.

3. The electrochemical system of claim 1, wherein the housing includes a deformable portion configured to be engaged by a user, the deformable portion being movable between an undeformed position in which the working electrode and the reference electrode are in the first configuration, and a deformed position in which the deformable portion urges the working electrode and the reference electrode into the second configuration.

4. The electrochemical sensing system of claim 3, further comprising:
a biasing member configured to urge the deformable portion from the deformed position to the undeformed position.

5. The electrochemical sensing system of claim 1, further comprising:
a penetrating member, the working electrode disposed on a first surface of the penetrating member, and the reference electrode disposed on a second surface of the penetrating member.

6. An electrochemical sensing system, comprising:
a working electrode including rhodium;
a reference electrode;
a housing configured for removably associating the working electrode and the reference electrode with the body of a user, the housing including a base portion and a movable portion being movable between and unassembled position and an assembled position by moving the movable portion with reference to the base portion;
an electrical circuit disposed in the base portion of the housing and configured to be electronically coupled to the working electrode and the reference electrode, the electrical circuit operable to: (a) bias the working electrode at a voltage of less than about 0.4 V such that a target analyte decomposes, and (b) measure a current corresponding to the concentration of the target analyte; and
a communication module disposed in the movable portion and configured to be electrically coupled to the electrical circuit
the working electrode and the reference electrode movable between a first configuration in which the working electrode and the reference electrode are disposed substantially inside the housing, and a second configuration in which at least a portion of the working electrode and a portion of the reference electrode are disposed outside the housing.

7. The electrochemical sensing system of claim 6, wherein the working electrode and the reference electrode are moved from the first configuration to the second configuration by moving the housing between the unassembled position and the assembled position.

8. The electrochemical sensing system of claim 6, wherein the communication module is configured to at least one of display a concentration of the target analyte, and communicate data between the electrical circuit and an external device.

9. An electrochemical sensing system, comprising:
a housing configured to be removably associated with a user;
a working electrode at least partially disposed in the housing, at least a portion of the working electrode including rhodium, palladium, gold, or platinum;
a synthetic redox-active receptor disposed on the working electrode and configured to move between different electronic states, the synthetic redox-active receptor further configured to change its reduction potential upon binding the target analyte such that the target analyte does not decompose;
a reference electrode at least partially disposed in the housing;
an electrical circuit electronically coupled to the working electrode and the reference electrode, the electrical circuit operable to: (a) bias the working electrode at a voltage in the range of about −0.6 V to about 0.4 V such that the working electrode dontates an electron to the synthetic redox-active receptor and moves the synthetic redox-active receptor into a more reduced state, and (b) measure a current corresponding to the concentration of the target analyte; and
a communication module coupled to the electrical circuit and configured to at least one of display a concentration of the target analyte, and communicate data between the electrical circuit and an external device, the data including the concentration of the target analyte,
the working electrode and the reference electrode movable between a first configuration in which the working electrode and the reference electrode are disposed substantially inside the housing, and a second configuration in which at least a portion of the working electrode and at least a portion of the reference electrode are disposed outside the housing.

10. The electrochemical sensing system of claim 9, wherein the synthetic redox-active receptor includes a viologen.

11. The electrochemical sensing system of claim 9, wherein the synthetic redox-active receptor includes a conjugated pyridinium.

12. The electrochemical sensing system of claim 9, wherein the synthetic redox-active receptor includes a boronic acid.

13. The electrochemical sensing system of claim 9, wherein the target analyte is glucose.

14. The electrochemical sensing system of claim 9, wherein the target analyte is lactate.
